(19) United States
(12) Patent Application Publication
(10) Pub. No.: US 2016/0120176 A1
(43) Pub. Date: May 5, 2016

(54) ANTIMICROBIAL FOAMS AND METHODS OF MAKING SAME

(71) Applicant: 3M INNOVATIVE PROPERTIES COMPANY, St. Paul, MN (US)

(72) Inventors: Shilpi K. Sanghi, Stanford, CA (US); Mahfuza B. Ali, Mendota Heights, MN (US)

(73) Assignee: 3M INNOVATIVE PROPERTIES COMPANY, St. Paul, MN (US)

(21) Appl. No.: 14/893,574

(22) PCT Filed: Jun. 25, 2014
(86) PCT No.: PCT/US14/44118

§ 371 Date: Nov. 24, 2015

Related U.S. Application Data
(60) Provisional application No. 61/841,705, filed on Jul. 1, 2013.

(51) Int. Cl. A01N 25/16; A01N 33/12; A61L 15/42; A01N 55/00

(52) U.S. Cl. CPC A01N 25/16; A01N 55/00; A01N 33/12; A61L 15/425; A61L 2300/404

(57) ABSTRACT

Antimicrobial foams and methods of making same. The antimicrobial foams can include a polymeric foam having a wet-out time of at least 250 sec; and a coating comprising an antimicrobial material. The antimicrobial material can include a quaternary ammonium compound, and the antimicrobial material can be covalently bound to the foam. The antimicrobial foam can include at least 0.25 parts by weight of quarternary ammonium compound per 100 parts by dry weight of the foam. The method can include providing the polymeric foam and an antimicrobial material comprising a quaternary ammonium precursor. The method can further include combining the foam and the quaternary ammonium precursor to form a combination; and heating the combination to a temperature of at least 40° C. to form an antimicrobial foam comprising a quaternary ammonium compound covalently bound to the foam.

### FIELD

The present disclosure generally relates to antimicrobial foams and compression dressings comprising same.

### BACKGROUND

Compression bandages are known for use in the treatment of edema, as well as venous and lymphatic disorders, e.g., of the lower limbs. Areas where compression bandages are considered particularly useful are the management and treatment of chronic wounds, such as venous leg ulcers.

Some existing venous leg ulcer treatments include application of a 2 to 4 layer compression bandage, whereby the concept of such multi-layer bandaging involves a combination of different types of bandage layers in order to apply pressure in layers (giving an accumulation of pressure) and to provide sustained compression together with rigidity. One existing bandage system employs a four-layer system including an inner layer of absorbent orthopedic wool, a second layer crepe bandage, a third layer of light compression bandage and a fourth layer of self-adherent (cohesive) flexible bandage.

When a compression bandage is left in position (e.g., wrapped over a limb) over a period of time (e.g., 1 day to 14 days), it can become malodorous. Odor can carry a social stigma and can cause embarrassment and isolation, can contribute to depression, and can generally impact quality of life. Venous leg ulcers are wounds that are often associated with odor. Odor under compression bandages can result from a variety of factors, such as sweat; dry, cracked or fissured skin; and/or normal skin micro flora. Sweat, skin flora, and/or exudate from a wound or cracked skin can lead to bacteria on the skin and in the bandage. These bacteria can secrete/excrete enzymes through normal metabolic activity, which in turn can generate odor-causing compounds, such as organic fatty acids. Charcoal and cyclodextrin can be used to absorb such fatty acids to control odor.

### SUMMARY

Antimicrobials can prevent growth of bacteria, which can cease or suppress metabolism, and therefore, cease or suppress the generation of the subsequent malodorous fatty acids. Antimicrobials can be classified into two types; leachable (e.g., silver (Ag), chlorhexidine gluconate (CHG), and polyhexamethylene biguanide (PHMB)) and non-leachable (e.g., covalently-bound quaternary amines). In some cases, leachable antimicrobials can be associated with concerns of generating adaptive organisms and toxicity by leaching harmful chemicals into the environment.

The present disclosure generally relates to antimicrobial foams and compression dressings or bandages comprising same, and particularly, the present disclosure relates to antimicrobial foams comprising non-leachable (i.e., covalently-bound) quaternary ammonium-based compounds.

Some aspect of the present disclosure provide an antimicrobial foam. The antimicrobial foam can include a polymeric foam, the foam having a wet-out time of at least 250 sec; and a coating comprising an antimicrobial material. The antimicrobial material can include a quaternary ammonium compound, and the antimicrobial material can be covalently bound to the foam. The antimicrobial foam can include at least 0.25 parts by weight of quarternary ammonium compound per 100 parts by dry weight of the foam.

Some aspects of the present disclosure provide a method of making an antimicrobial foam. The method can include providing a polymeric foam, the foam having a wet-out time of at least 250 sec. The method can further include providing an antimicrobial material comprising a quaternary ammonium precursor. The method can further include combining the foam and the quaternary ammonium precursor to form a combination; and heating the combination to a temperature of at least 40° C. to form an antimicrobial foam comprising a quaternary ammonium compound covalently bound to the foam.

Other features and aspects of the present disclosure will become apparent by consideration of the detailed description and accompanying drawings.

### BRIEF DESCRIPTION OF THE DRAWINGS

FIG. 1 is a perspective view of a compression dressing according to one embodiment of the present disclosure, the compression dressing including an antimicrobial foam according to one embodiment of the present disclosure. The compression dressing can form an inner bandage or comfort layer of a compression bandage system.

FIG. 2 is a perspective view of an outer bandage that can be used in combination with the compression dressing (i.e., inner bandage) of FIG. 1 to form a compression bandage system.

FIG. 3 is a schematic diagram showing the chemical structure of an antimicrobial foam according to one embodiment of the present disclosure.

FIG. 4 is a schematic diagram showing the chemical structure of an antimicrobial foam according to another embodiment of the present disclosure.

FIG. 5 is a schematic diagram showing the chemical structure of an antimicrobial foam according to another embodiment of the present disclosure.

### DETAILED DESCRIPTION

Before any embodiments of the present disclosure are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “affixed,” and “coupled” and variations thereof are used broadly and encompass both direct and indirect affixations and couplings. It is to be understood that other embodiments may be utilized, and structural or logical changes may be made without departing from the scope of the present disclosure.

The present disclosure generally relates to antimicrobial foams and compression dressings or bandages comprising same. Particularly, the present disclosure relates to antimicrobial foams comprising non-leachable quaternary ammonium-based compounds. Generally, “non-leaching” or “non-leachable” antimicrobials refers to antimicrobial materials that are covalently bonded to the chemical structure making up the foam.

The term “foam” and the phrase “polymeric foam” each generally refer to a polymeric material containing open and/or closed cells dispersed throughout its mass. In some embodiments, foams of the present disclosure can include polymeric foams containing open cells. Suitable foams can include flexible, resilient foams.

Foams used in the antimicrobial foams and compression dressings of the present disclosure are generally formed of at least one of polyurethane, polyester, and polyether. The antimicrobial materials of the present disclosure can be applied to the foams after formation of the foam, while still allowing for the antimicrobial material to be covalently bonded to the foam. As a result, the foams employed in the antimicrobial foams and compression dressings of the present disclosure can be coated with the antimicrobial material such that the antimicrobial material covalently bonds with the foam. For example, the antimicrobial material can include one or more moieties configured to chemically react (i.e., to form a covalent bond) with unreacted hydroxyl, isocyanate groups and/or amine groups in the foam. Such unreacted hydroxyl, isocyanate groups and/or amine groups may be present on an outer surface of the foam, and/or may be present at least partially within the volume of the foam. Polyurethane foams may include hydroxyl, isocyanate, and/or amine groups with which the antimicrobial material can react; polyester foams may include hydroxyl groups with which the antimicrobial material can react; and polyether foams may include hydroxyl groups with which the antimicrobial material can react.

The term “coating” is generally used to refer to a material that is applied to the foam after formation of the foam, rather than during formation of the foam, and is not limited to materials that may end up being present only on the outermost surface of the foam, but can also refer to materials that may have been taken up at least partially into the internal volume of the foam, e.g., to coat interstices of the foam.

Antimicrobial materials of the present disclosure can include quaternary ammonium compounds. Quaternary ammonium compounds generally act by disrupting a cell membrane. Quaternary ammonium compounds, and particularly, quaternary ammonium compounds comprising an alkyl chain, can bind by ionic and hydrophobic interactions to the surface of microbial membranes, such that the cationic head is facing outwards and the hydrophobic tail is inserted into the lipid bilayer of the microbial membrane. This can cause membrane damage and leakage of intracellular constituents from the cell, ultimately resulting in cell death.

In some embodiments, quaternary ammonium compounds of the present disclosure can include one or more hydroxy- and/or alkoxy-modified silanes and/or hydroxy- and/or alkoxy-modfied esters. As described in greater detail below, in some embodiments, the quaternary ammonium-based antimicrobial materials of the present disclosure can include (i) a silane-based quaternary ammonium compound, (ii) a polyacrylate quaternary ammonium compound (e.g., a copolymer of one or more quaternary ammonium-containing monomers and one or more monomers that have, e.g., at least one of unreacted hydroxyl (—OH) groups, alkoxyl (—OR) groups, carboxyl (—COOH) groups, isocyanate (—NCO) groups, and amine (—NH₂) groups capable of reacting with the foam), or (iii) a combination thereof. Alkoxysilanes in silane-based quaternary ammonium compounds generally hydrolyze to silanols which can then react with the foam, e.g., to form a silyl ether bond, if reacting with a hydroxyl group in the foam. The silane-based quaternary ammonium compounds can undergo two reactions simultaneously: (i) self condensation reaction forming siloxane groups and leading to polymerization, and (ii) reaction with a moiety of the foam (e.g., a hydroxyl (—OH) group, an isocyanate (—NCO) group, and/or an amine (—NH₂) group) to form covalent attachment with the foam. In some embodiments, monomeric acrylate quaternary ammonium compounds are copolymerized with other monomers that have a pendant —OH group or another moiety (e.g., those listed above) that can react with foam. The resulting copolymer has one or more unreacted pendant groups that can react with the foam, as described in greater detail below.

Generally, the type of bond that is formed between the polyacrylate quaternary ammonium compound and the foam can be dependent upon the moieties that are present as pendant chain groups from the polyacrylate, as well as the available moieties in the foam. As described in greater detail below, in some embodiments, the polyacrylate can be formed from repeat units that include different monomers, where one or more of the monomers includes a quaternary ammonium, e.g., in a pendant chain group that extends from the polyacrylate backbone.

The antimicrobial material can covalently attach to the foam, and in some embodiments, can further bond to other adjacent antimicrobial materials (i.e., quaternary ammonium compounds) to form crosslinks with other quaternary ammonium compounds that may, in turn, be covalently bonded to the foam.

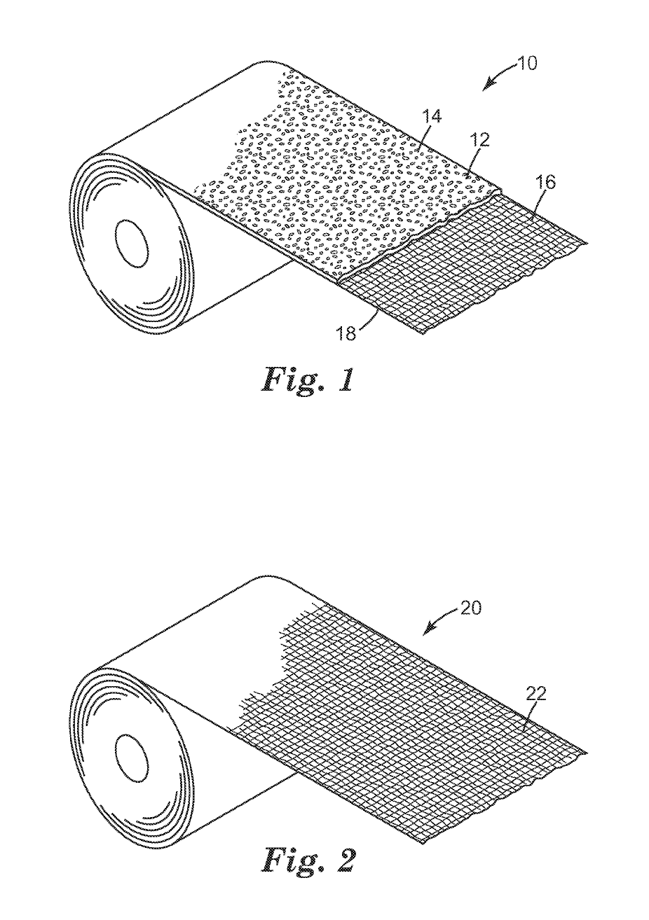

In some embodiments, the antimicrobial foams of the present disclosure can form at least a portion of a comfort layer for a compression dressing or bandage system. For example, as described below with reference to FIGS. 1 and 2, some bandage systems employ an inner, comfort layer that is positioned directly adjacent the skin in use, and an outer (e.g., self-adhesive) layer that functions to add compression and to hold the entire bandage system in place. Such a comfort layer can include a foam that is positioned directly on the skin, and which can be the source of malodor.

In some embodiments, the antimicrobial foams of the present disclosure can form at least a portion of a compression dressing, a wound dressing, or a combination thereof.

Generally, the antimicrobial foams of the present disclosure have low absorbency (and generally have a wet-out time of at least 250 seconds, in some embodiments, at least 300 seconds, in some embodiments, at least 500 seconds, in some embodiments, at least 750 seconds, in some embodiments, at least 1000 seconds. The following test method can be used to determine the wet-out time of a given foam:
Begin with a foam sample of an approximate size of 2 inches (about 5 cm)×2 inches (about 5 cm). Draw 1 mL of demineralized water into a pipet. Lay the sample flat and bring the pipet close to the horizontal surface of the sample. Measure the time (in seconds) that a 1 mL drop of water from the pipet needs to penetrate totally into the foam. The penetration is determined visually. Report the wet-out time in seconds (or seconds/mL).

In some embodiments, the antimicrobial foams of the present disclosure can be made by combining the antimicrobial material (e.g., a quaternary ammonium compound precursor) and the foam (i.e., after formation of the foam) and heating the combination for a period of time at a temperature that causes the quaternary ammonium compound precursor to chemically react (i.e., covalently bond) with the foam. In some embodiments, the quaternary ammonium compound precursor can be monomeric, oligomeric, polymeric, or a combination thereof.

The temperature at which the combination needs to be heated can vary, depending on the duration of the heating step. In addition, the time and temperature required for the heating step can change, depending on whether a dried foam is used. In embodiments in which polyacrylate quaternary ammonium compound monomers or oligomers are employed as the antimicrobial material precursor (or quaternary ammonium compound precursor), higher temperatures are generally necessary to cause both polymerization of the polyacrylate as well as a reaction (i.e., formation of covalent bonds) between the polyacryate quaternary ammonium compound and the foam. However, it should be noted that enhanced reaction efficiencies and antimicrobial effectiveness can be achieved when the polyacrylate quaternary ammonium compound is polymerized prior to being reacted with the foam (i.e., prior to being placed in contact with, or applied to, the foam).

In some embodiments, the combination can be heated to a temperature of at least 40° C., in some embodiments, at least 50° C., in some embodiments, at least 75° C., in some embodiments, at least 100° C., in some embodiments, at least 105° C., in some embodiments, at least 150° C., and in some embodiments, at least 200° C. In some embodiments, the combination can be heated for a period of at least 1 min., in some embodiments, at least 5 min., in some embodiments, at least 15 min, in some embodiments, at least 20 min, in some embodiments, at least 30 min., in some embodiments, at least 1 hour, in some embodiments, at least 1.5 hours, in some embodiments, at least 2 hours, in some embodiments, at least 5 hours, in some embodiments, at least 10 hours, in some embodiments, at least 15 hours, in some embodiments, at least 20 hours, and in some embodiments, at least 24 hours.

In some embodiments (e.g., when a dried foam is combined with a quaternary ammonium compound of the present disclosure), the antimicrobial foam of the present disclosure can be formed by heating the combination at 65° C. for 15 hours; in some embodiments, at 75° C. for 1.5 hours; in some embodiments, at 105° C. for 2 hours; in some embodiments, at 150° C. for 1 min.; and in some embodiments, at 200° C. for 1 min.

In some embodiments, the foam and the antimicrobial material can be combined and then heated (i.e., the combination can be heated after the antimicrobial material has been applied to the foam). In some embodiments, the foam can be heated and/or the antimicrobial material (e.g., a solution thereof) can be heated prior to, during, and/or after combining the foam and the antimicrobial material, such that at least the combination is heated. As a result, in some embodiments, combining the foam and the antimicrobial material and heating the combination can occur at least partially simultaneously.

Combining the foam and the antimicrobial material can be accomplished by applying the antimicrobial material to the foam (or coating the foam with the antimicrobial material) and can include at least one of dipping (or dip-coating), spraying (or spray-coating), curtain coating, brushing, swabbing, padding, and a combination thereof. The resulting coated product can then be heated for a period of time sufficient to covalently bond the antimicrobial material and the foam. Zone of inhibition tests can be used to confirm that the antimicrobial is not leaching away from foam.

The antimicrobial foams of the present disclosure are particularly suited to reduce microbial growth, e.g., microbes that can cause malodor. As a result, the antimicrobial foams of the present disclosure include sufficient antimicrobial material to substantially inhibit microbial growth. In some embodiments, substantially reducing microbial growth can include exhibiting at least a 1 log reduction, in some embodiments, at least a 2 log reduction, in some embodiments, at least a 3 log reduction, in some embodiments, at least a 5 log reduction, and in some embodiments, at least a 6 log reduction in either gram positive or gram negative bacteria, e.g., when tested pursuant to ASTM E2149-10 and compared to an untreated/uncoated control, as exhibited in the Examples below.

In some embodiments, sufficient antimicrobial material in the resulting antimicrobial foam of the present disclosure can include at least (or more than) 0.25 parts by weight of quaternary ammonium compound per 100 parts by dry weight of the foam (0.25%); in some embodiments, at least (or more than) 0.5 parts by weight of quaternary ammonium compound per 100 parts by dry weight of the foam (0.5%); in some embodiments, at least (or more than) 1 part by weight of quarternary ammonium compound per 100 parts by dry weight of the foam (1 wt %); in some embodiments, at least (or more than) 2 parts by weight of quaternary ammonium compound per 100 parts by dry weight of the foam (2 wt %); in some embodiments, at least (or more than) 5 parts by weight of quaternary ammonium compound per 100 parts by dry weight of the foam (5 wt %); in some embodiments, at least (or more than) 10 parts by weight of quaternary ammonium compound per 100 parts by dry weight of the foam (10 wt %); in some embodiments, at least (or more than) 20 parts by weight of quaternary ammonium compound per 100 parts by dry weight of the foam (20 wt %); in some embodiments, at least (or more than) 25 parts by weight of quaternary ammonium compound per 100 parts by dry weight of the foam (25 wt %); in some embodiments, at least (or more than) 30 parts by weight of quaternary ammonium compound per 100 parts by dry weight of the foam (30 wt %); and in some embodiments, at least (or more than) 40 parts by weight of quaternary ammonium compound per 100 parts by dry weight of the foam (40 wt %).

In some embodiments, a compression dressing or bandage system of the present disclosure can include inner and outer bandages. The inner bandage can be an inner skin-facing, elongated, elastic bandage and can include an elongated, elastic substrate and an elongated foam layer affixed to a face of the substrate. In some embodiments, the elongated foam layer can extend 33% or more across the face of the substrate in a transverse direction, and 67% or more across the face of the substrate in a longitudinal direction. The outer bandage can be an outer, elongated, self-adhering elastic bandage having a compressive force when extended.

Some embodiments of compression bandage systems of the present disclosure include an inner skin facing, elongated, elastic bandage (or comfort layer) 10 (as exemplified in FIG. 1 and described in greater detail below) and an outer, elongated, self-adhering, elastic compression bandage 20 (as exemplified in FIG. 2 and described in greater detail below). Each bandage can be sufficiently elongated along a longitudinal direction so as to be capable of being wound two or more turns (more suitably five or more turns) about a limb of a patient.

As shown in FIG. 1, the inner, skin facing elongated, elastic bandage 10 includes an elongated, elastic substrate 16 and an elongated layer of foam 12. The foam layer 12 can be or include one or more antimicrobial foams of the present disclosure. For example, in some embodiments, the foam 12 can include a plurality of layers and one or more of the layers of the foam 12 can include an antimicrobial foam of the present disclosure. Additionally, or alternatively, in some embodiments, the antimicrobial foam of the present disclosure can be employed along a portion of the length of the inner bandage 10, e.g., in a pattern (e.g., alternating) with non-antimicrobial foam, such that enough antimicrobial foam is present along the length of the inner bandage 10 to inhibit microbial growth and malodor formation.

As shown in FIG. 1, the foam layer 12 can be coupled to a face of said elastic substrate 16. In some embodiments, the foam layer 12 can extend 33% or more across the face of the substrate 16 in a transverse direction and 67% or more across the face of the substrate 16 in a longitudinal direction.

As shown in FIG. 2, the outer bandage 20 may suitably comprise a woven (e.g., knitted) or nonwoven bandage comprising generally a plurality of generally longitudinally extending elastic yarns in the woven, knitted or nonwoven structure 22. In some embodiments, the elastic structure 22 can be coated or impregnated with a polymer binder. For example, in some embodiments, the outer bandage 20 can include a plurality of generally longitudinally extending, (preferably partially extended) elastic yarns bound with a polymeric binder between two webs or bound with a polymeric binder on a web. In some embodiments, the polymeric binder is cohesive, so that the bandage is self-adherent (i.e. in use, the bandage will remain adhered to itself under elastic extension e.g., without the use of a fastening mechanism), but will not adhere to clothing, hair or skin. Accordingly, generally the top and bottom faces of the bandage comprise polymeric binder, e.g., where the polymeric binder generally extends throughout the thickness of the elastic structure 22.

Additional details of exemplary compression dressings or bandage systems of the present disclosure are described below and are further described in U.S. Pat. No. 7,854,716 (Schuren et al.), which is incorporated herein by reference.

### Silane-Based Antimicrobial Material

As described above, in some embodiments, the antimicrobial material can include a silane-based antimicrobial material, i.e., a silane-based quaternary ammonium compound, which can be monomeric or polymeric, as described below. Furthermore, as mentioned above, the silane-based quaternary ammonium compound can include a hydroxysilane (i.e., silanol) or an alkoxysilane (e.g., methoxysilane or ethoxysilane).

In some embodiments, the quaternary ammonium compound that is covalently bound to the foam is derived from a reaction between a quaternary ammonium compound precursor and at least one of an unreacted hydroxyl, isocyanate, and amine group of the foam. In some embodiments, the silane-based quaternary ammonium precursor can be a salt having formula I:

(R¹O)₃SiR²N⁺R³R⁴(CH₂)ₙCH₃X⁻ (I)
where:
R¹ is selected from H, CH₃, or C₂H₅,
R² has the formula CₘH₂ₘ₊₁, where m is an integer ranging from 1 to 4,
R₃ and R₄ could be H, CH₃, C₂H₅,
n is an integer ranging from 2 to 22, and
X is selected from Cl, Br, BF₄, N(SO₂CF₃)₂, O₃SCF₃, and O₃SC₄F₉.
   By way of example, in some embodiments, the silane-based quaternary ammonium precursor can be a quaternary ammonium chloride, such as 3-trimethoxysilylpropyldimethyloctadecyl ammonium chloride having structural formula II:

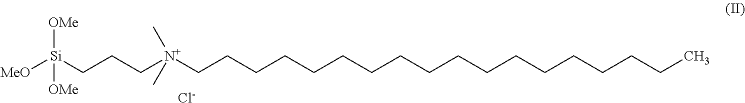 [SMILES: CCCCCCCCCCCCCCCCCC[N+](C)(C)CCC[Si](OC)(OC)OC ; [Cl-]]

In the presence of water, alkoxysilanes (e.g., methoxysilanes, as shown in Formula II) hydrolyze to silanols, that can undergo a condensation reaction to form a siloxane bond with other (adjacent) silane-based quaternary ammonium compounds, with elimination of water, to form a polymer. That is, the hydroxyl (—OH) group of silanols can react with each other (i.e., to form siloxane bonds) and polymerize and/or can attach covalently to the foam by reacting with unreacted hydroxyl groups, isocyanate groups, and/or amine groups on the substrate, as shown in Schemes A, B and C. Polyurethane foam can include available hydroxyl groups and isocyanate groups. The isocyanate groups may react with water to form amines in the polyurethane foam. Polyester foam can have unreacted available hydroxyl groups and/or carboxyl groups. For example, the carboxyl groups can form a silyl ester with a silanol from the silane-based quaternary ammonium compound. Polyether foam can include available hydroxyl groups. The silane-based quaternary ammonium compounds discussed herein can react with the hydroxyl groups, isocyanate groups and/or the amine groups of the foam according to any of Schemes A, B and C shown below. In addition, for example, the silane-based quaternary ammonium compound can react with carboxyl groups in the foam, as mentioned above. Scheme A can apply to polyurethane foams, polyester foams, and polyether foams, and Schemes B and C can apply to polyurethane foams.

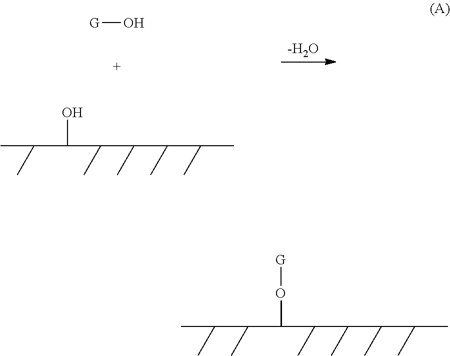

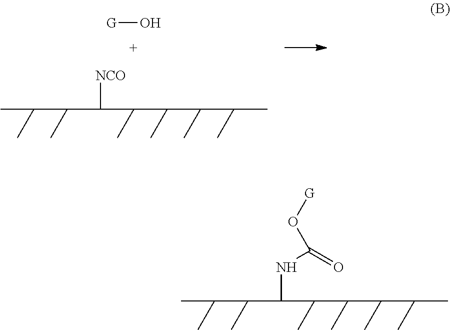

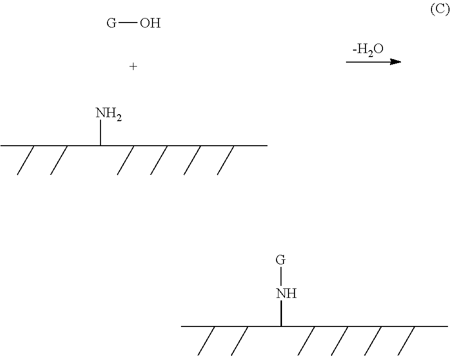

G-OH in Schemes A, B and C represents a compound containing silanol groups (e.g., that could have resulted from hydrolysis of Si—OR groups) or any monomer or polymer having a hydroxyl group (e.g., that could have resulted from hydrolysis of an alkoxyl group). That is, G-OH can represent a monomer having the formula II. Particular advantages in antimicrobial activity have been found in specific compounds of Formula I, e.g., where n is 18. Formula III below represents a specific example of Formula I, where n is 18 and R is selected from H, CH₃, or C₂H₅. Formula IV below represents an exemplary polymeric silane-based quaternary ammonium compound (i.e., precursor) that can be reacted with the foam, where the polymeric silane-based quaternary ammonium compound (i.e., precursor) includes monomers of Formula III that have been polymerized via siloxane bonds. Such polymerization can occur before, after and/or during reaction with the foam.

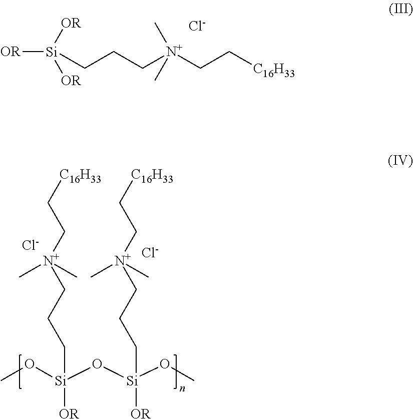

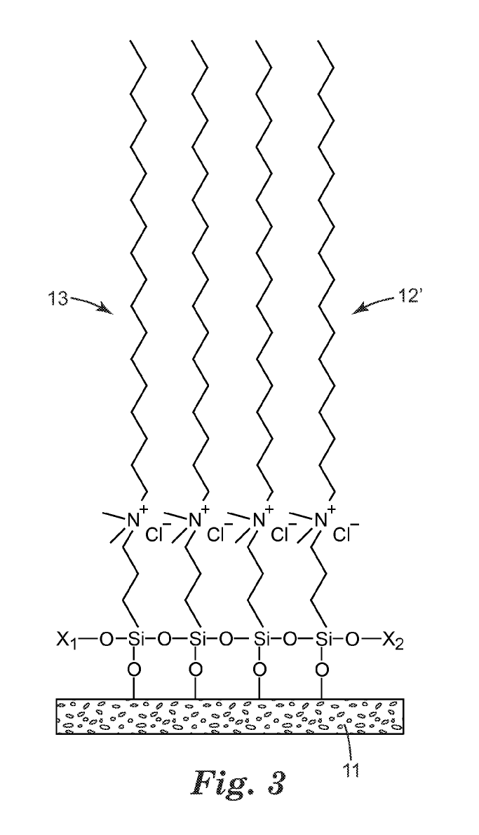

FIG. 3 schematically illustrates one example of an antimicrobial foam 12′ of the present disclosure, where 3-trimethoxysilylpropyldimethyloctadecyl ammonium chloride was reacted with hydroxyl groups in a foam 11 to form an antimicrobial material (i.e., a quaternary ammonium compound) 13 covalently bound to the foam 11. The antimicrobial foam 12′ can be employed in at least a portion of the foam layer 12 of the inner bandage 10 of FIG. 1. In FIG. 3, X₁ and X₂ can be a hydrogen, a methyl group, or can represent a continuation of the crosslinking with other silane-based quaternary ammonium compounds, as shown in FIG. 3 and Formula IV. As shown in FIG. 3, in some embodiments, quaternary ammonium compounds (i.e., silane-based quaternary ammonium compounds) of the present disclosure can be covalently bonded to the foam via a silyl ether bond.

### Polyacrylate-Based Antimicrobial Material

As described above, in some embodiments, the antimicrobial material can include a polyacrylate quaternary ammonium compound, which can include silane-based and non-silane-based quaternary ammonium compounds, and may include additional functional groups configured to react with the foam. In some embodiments, such a polyacrylate quaternary ammonium compound can include a polyacrylate quaternary ammonium oligomer, polymer, or a combination thereof. In embodiments in which monomers or oligomers that are used to form the polyacrylate quaternary ammonium polymer are combined with the foam, the combination may need to be heated to higher temperatures, and an initiator for initiating polymerization may need to be added, to accomplish both polymerization of the polyacrylate as well as reaction of the polyacrylate quaternary ammonium compound with the foam. In such embodiments, heating can initiate (i) polymerization of acrylate groups of the polyacrylate quaternary ammonium compound to form the polyacrylate quaternary ammonium polymer, and (ii) reaction of the polyacrylate quaternary ammonium compound with the foam, which, in some embodiments, can occur simultaneously with polymerization. However, as mentioned above, enhanced reaction efficiencies and antimicrobial effectiveness can be achieved when the polyacrylate quaternary ammonium compound is polymerized prior to being reacted with the foam (i.e., prior to being placed in contact with, or applied to, the foam).

In some embodiments, the polyacrylate quaternary ammonium compound (i.e., the precursor that is reacted with the foam) can be derived from quaternary amine-functionalized (or quaternary ammonium) ethylenically unsaturated monomers. That is, in some embodiments, the polyacrylate quaternary ammonium compound can include one or more monomers having formula V:

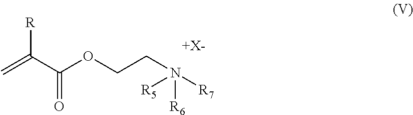

where:
R is selected from H and CH₃;
R⁵ and R⁶ are each selected from CH₃ and C₂H₅;
R⁷ is selected from CH₃, C₄H₉, C₆H₁₃, C₁₀H₂₁, C₁₂H₂₅, C₁₆H₃₃, C₁₈H₃₇, C₂₀H₄₁, and C₂₂H₄₅; and
X is selected from Cl, Br, BF₄, N(SO₂CF₃)₂, O₃SCF₃, and O₃SC₄F₉.

Specific examples of suitable quaternary amine-functionalized ethylenically unsaturated monomers include dimethylhexadecylammoniumethylacrylate halides (DMAEA-C₁₆ halides; e.g., dimethylhexadecylammoniumethylacrylate bromides (DMAEA-C₁₆Br)), dimethyl-hexadecylammoniumethylmethacrylate halides (DMAEMA-C₁₆ halides; e.g., dimethyl-hexadecylammoniumethylmethacrylate bromides (DMAEMA-C₁₆Br)), and derivatives thereof. Examples of suitable derivatives of DMAEA-C₁₆ halides and DMAEMA-C₁₆ halides include derivatives of DMAEA-C₁₆Br and DMAEMA-C₁₆Br, as described below, but it should be understood that similar derivatives of other DMAEA-C₁₆ halides and DMAEMA-C₁₆ halides are within the spirit and scope of the present disclosure, and one of ordinary skill in the art would understand how to extend the description below to such other halides.

Suitable derivatives of DMAEMA-C₁₆Br have the structural formula of formula VI:

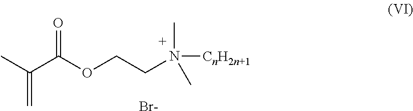

where suitable values for “n” range from 8-22, with particularly suitable values for “n” ranging from about 10-16. Such polymer-chain lengths allow the DMAEMA derivative to move enough within the cross-linked matrix while also preventing the DMAEMA derivative from phase separating from the resulting cross-linked matrix.

DMAEMA-C₁₆Br and its derivatives may be formed by combining dimethylaminoethylmethacrylate salt, acetone, 1-bromohexadecane, and optionally, an antioxidant. The mixture may be stirred for about 16 hours at about 35° C. and then allowed to cool to room temperature. The resulting white solid precipitate may then be isolated by filtration, washed with cold ethyl acetate, and dried under vacuum at 40° C.

Similarly, DMAEA-C₁₆Br and its derivatives may be formed by combining dimethylaminoethylacrylate, of acetone, 1-bromohexadecane, and optionally, an antioxidant. The mixture may be stirred for 24 hours at 35° C., and then allowed to cool to room temperature. The acetone may then be removed by rotary evaporation under vacuum at 40° C. The resulting solids may then be washed with cold ethyl acetate and dried under vacuum at 40° C.

In some embodiments, the polyacrylate quaternary ammonium compound (i.e., the precursor that is reacted with the foam) includes a quaternary ammonium compound (i.e., the antimicrobial moiety) and a moiety that can react with the foam. Such a polyacrylate quaternary ammonium compound can be obtained by copolymerization of acrylate quaternary ammonium compound monomers (such as those shown in Formulas V and VI) with other monomers having acrylate functionality as well as a moiety capable of covalently bonding to the foam. That is, in some embodiments, the polyacrylate quaternary ammonium compound can be formed by copolymerizing two or more monomers: (i) a monomer that contains a quaternary ammonium compound and (ii) at least one monomer that contains a pendant group capable of reacting with the foam to form a covalent bond (e.g., a pendant hydroxyl group, a pendant alkoxyl group (e.g., methoxyl), a pendant trimethoxy silyl group, a pendant amine group, a pendant isocyante group, and/or a pendant carboxyl group), and/or a pendant group that generates a moiety (e.g., a hydroxyl group) in situ that can react with the foam. Particular antimicrobial effectiveness has been demonstrated using polyacrylate quaternary ammonium compounds formed by copolymerizing three monomers, e.g., as shown in Formulas VII and VIII, and exemplified in the Examples. Additional details of polyacrylate quaternary ammonium polymers that can be employed in antimicrobial foams and compression dressings of the present disclosure can be found in PCT Publication WO 2011/150103 (Attorney Docket No. 67609WO003), which is incorporated herein by reference.

For example, some polyacrylate quaternary ammonium compounds (i.e., polymeric precursors) of the present disclosure can have the copolymeric structural formula of Formula VII:

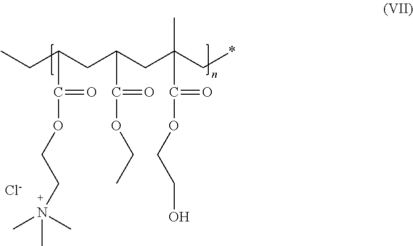

As shown, a copolymer of Formula VII can include a pendant hydroxyl (—OH) group that can be reacted with at least one of an unreacted hydroxyl group, isocyanate group and/or amine group of the foam, as detailed above in Schemes A, B and C.

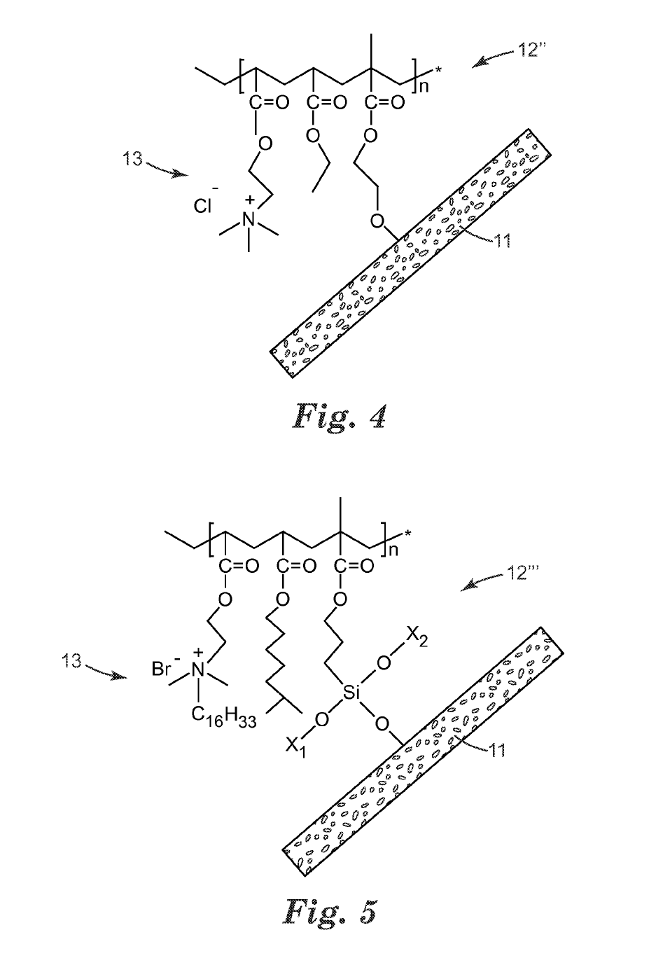

FIG. 4 schematically illustrates another example of an antimicrobial foam 12″ of the present disclosure, where a copolymer of Formula VII was reacted with hydroxyl groups in a foam 11 to form an antimicrobial material (i.e., a quaternary ammonium compound) 13 covalently bound to the foam 11. In some embodiments, the antimicrobial foam 12″ can be employed in at least a portion of the foam layer 12 of the inner bandage 10 of FIG. 1. As shown in FIG. 4, in some embodiments, polyacrylate quaternary ammonium compounds of the present disclosure can be covalently bonded to the foam via an ether bond.

By way of example, some polyacrylate quaternary ammonium compounds of the present disclosure can have the copolymeric structural formula of Formula VIII:

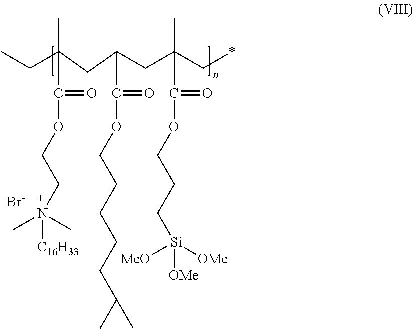

In some embodiments, polyacrylate quaternary ammonium compounds of the present disclosure can include one or more pendant (—OR) group that can be reacted with the foam. By way of example, copolymers of Formula VIII include a pendant methoxy (—OMe or —OCH₃) group that can hydrolyze to hydroxyl groups, which can (i) polymerize with other adjacent monomers, oligomers or polymers (e.g., via siloxane bonds, as shown in Formula IV) and/or (ii) react with at least one of an unreacted hydroxyl, isocyanate, and amine group of the foam, as detailed above in Schemes A, B and C.

FIG. 5 schematically illustrates another example of an antimicrobial foam 12′″ of the present disclosure, where a copolymer of Formula VIII was reacted with hydroxyl groups in a foam 11 to form an antimicrobial material (i.e., a quaternary ammonium compound) 13 covalently bound to the foam 11. The antimicrobial foam 12′″ can be employed in at least a portion of the foam layer 12 of the inner bandage 10 of FIG. 1. As shown in FIG. 5, in some embodiments, polyacrylate quaternary ammonium compounds of the present disclosure can be covalently bonded to the foam via a silyl ether bond.

In some embodiments, copolymers of Formula VII or VIII can be formed by reacting monomers of formula V or VI with other ethylenically unsaturated monomers, as described in the Examples section below. That is, in some embodiments, the polyacrylate quaternary ammonium compound that is combined with the foam can be derived from a combination of the monomers (or oligomers or polymers thereof) of Formulas V, VI, and other monomers having an acrylate moiety that also have one or more moieties (e.g., a hydroxyl group, an alkoxyl group that can hydrolyze to a hydroxyl group, a carboxyl group, an isocyanate group, and/or an amine group) that can react with the foam. For example, the polyacrylate quaternary ammonium compound can form at least one of an ether bond, a silyl ether bond, an ester bond, a silyl ester bond, a urethane bond, a silyl urethane bond, an amide bond, and a silyl amide bond with the foam. Furthermore, in some embodiments of the present disclosure, the antimicrobial material can include one or both of the silane-based quaternary ammonium compounds of Formulas I-IV and the polyacrylate quaternary ammonium compounds (or oligomers or polymers thereof) of Formulas VII or VIII. That is, in some embodiments, a mixture or combination of the various quaternary ammonium compounds described herein can be applied to the foam and reacted with the foam to form an antimicrobial foam having a non-leaching antimicrobial material.

While the antimicrobial foams of the present disclosure are described above as being able to form at least a portion of the foam layer 12 of the comfort layer of the compression bandage system shown in FIGS. 1 and 2, it should be understood that the antimicrobial foams of the present disclosure can form at least a portion of a foam or comfort layer of a variety of compression dressings or bandage systems, and the specific bandage system of FIGS. 1 and 2 is illustrated and described by way of example only.

The Examples section below exemplifies the reduction of microbial growth and odor of antimicrobial foams of the present disclosure formed with various exemplary quaternary ammonium compounds of the present disclosure. Particularly, S. epidermidis and P. aeruginosa were tested, which are representative gram positive and gram negative bacteria, respectively, and which are believed to contribute to wound odor.

### Compression Dressings

Compression dressings or bandage systems as can be used to provide a desired therapeutic effect for extended periods of time. Such compression bandage systems can be used such that the inner bandage 10 of FIG. 1 faces the skin and the outer bandage 20 of FIG. 2 overlies the inner bandage 10. The inner skin facing, elongated, elastic bandage can adhere to the outer, elongated, self-adhering elastic bandage. In use, the inner skin facing, elongated, elastic bandage can adhere to the outer, elongated, self-adhering elastic bandage under elastic extension without the use of a fastening mechanism. In some embodiments, the system is free of any additional elongated bandages.

The inner bandage can comprise an outer face not affixed to the foam layer, wherein the exposed face comprises a self-adhering material such as a self-adhering elastomeric material. The compression bandage system can be designed such that the inner skin facing, elongated, elastic bandage provides less compression than the outer, elongated, self-adhering elastic bandage when extended. The elongated foam layer can be coextensive with the elongated, elastic substrate. The compression bandage system can further comprise a non-elongated wound dressing or plaster. The outer bandage can have a stretch capability of 75% at most in the longitudinal direction, and/or a recovery-of-stretch capability of at least 85% in the longitudinal direction. The inner bandage can have a stretch capability of less than 75% in the longitudinal direction, and/or a recovery-of-stretch capability of at least 80% in the longitudinal direction. The foam layer can have a thickness greater than 1.6 mm.

Surprisingly, it has been found that through the provision of a compression bandage system comprising: (a) an inner skin-facing, elongated, elastic bandage comprising an elongated, elastic substrate and an elongated layer of foam, said foam layer being affixed to a face of the substrate and extending 33% or more across the face of substrate in transverse direction and 67% or more in longitudinal direction; and (b) an outer, elongated, self-adhering elastic bandage which has a compressive force when extended, it is possible to provide a compression bandage system which is easy to apply and provides a desired therapeutic effect for extended periods of time.

In some embodiments, in use, the compression bandage system comprises: a) an inner skin facing, elongated, elastic bandage having inner and outer faces and comprising: (i) an elongated, elastic substrate having first and second faces, the second face comprising a self-adhering material, and (ii) an elongated layer of foam, said foam layer being affixed to the first face of said substrate and extending 33% or more across said first face of substrate in transverse direction and 67% or more across said first face of substrate in longitudinal direction, the foam layer having an exposed face not affixed to the first face of said substrate and not comprising a self-adhering material, the inner face of the inner bandage comprising the exposed face of the foam layer, and the outer face of the inner bandage comprising the second face of the elongated, elastic substrate; and b) an outer, elongated, self-adhering elastic bandage; said bandage having a compressive force when extended; wherein, in use, said outer bandage overlies the inner bandage, and said inner face of the inner bandage faces the skin, and the outer face of the inner bandage faces said outer bandage, wherein the inner and outer bandages are configured and adapted such that in use said bandages remain adhered to one another under elastic extension without the use of a fastening mechanism, and wherein the bandage system is free of any additional elongated bandages.

In some embodiments, in use, the compression bandage system comprises: a) an inner skin facing, elongated, elastic bandage comprising: (i) an elongated, elastic substrate, and (ii) an elongated layer of foam, said foam layer being affixed to a face of said substrate and extending 33% or more across said face of substrate in transverse direction and 67% or more across said face of substrate in longitudinal direction; and b) an outer, elongated, self-adhering elastic bandage; said bandage having a compressive force when extended; wherein, in use, said foam layer of the inner bandage faces the skin and the outer bandage overlies the inner bandage, wherein the inner and outer bandages are configured and adapted such that in use said bandages remain adhered to one another under elastic extension without the use of a fastening mechanism, wherein the bandage system is free of any additional elongated bandages, and wherein the elongated, elastic substrate of the inner bandage, when extended, provides less compression than the outer bandage when extended.

In some embodiments, in use, the compression bandage system comprises: a) an inner skin facing, elongated, elastic bandage having inner and outer faces and comprising: (i) an elongated, elastic substrate having first and second faces, the second face comprising a self-adhering material, and (ii) an elongated layer of foam, said foam layer being affixed to the first face of said substrate and extending 33% or more across said first face of substrate in transverse direction and 67% or more across said first face of substrate in longitudinal direction, the foam layer having an exposed face not affixed to the first face of said substrate and not comprising a self-adhering material, the inner face of the inner bandage comprising the exposed face of the foam layer, and the outer face of the inner bandage comprising the second face of the elongated, elastic substrate; and b) an outer, elongated, self-adhering elastic bandage; said bandage having a compressive force when extended; wherein, in use, said outer bandage overlies the inner bandage, and said inner face of the inner bandage faces the skin, and the outer face of the inner bandage faces said outer bandage, wherein the inner and outer bandages are configured and adapted such that in use said bandages remain adhered to one another under elastic extension without the use of a fastening mechanism, and wherein the bandage system is free of any additional elongated bandages, and the inner bandage when extended provides less compression than the outer bandage when extended.

The term “elongated bandage” as used herein is generally understood to mean that the bandage is sufficiently elongated so as to be capable of being wound 2 turns or more (more suitably 5 turns or more) about a limb of a patient.

In use, the foam layer of the inner bandage faces the skin with the outer bandage overlying the inner bandage. It has been found that due to the elasticity of the inner bandage substrate as well as advantageous interfacing between it and the outer bandage upon application, the skin-facing foam layer, in particular the exposed face of the foam layer facing directly towards the skin of the patient, demonstrates a particularly desirable and effective fastening onto the skin of the patient, which minimizes of tendency of the bandage system towards slippage after application.

In some embodiments, it can be preferable to include an outer, elastic, compression bandage having a stretch capability in the longitudinal direction of not more than 75% (more preferably not more than 65%, most preferably not more than 55%). With such outer compression bandages, it is relatively easy, in particular for inexperienced staff, to apply the bandage at the desired therapeutic pressure, for example by applying the outer bandage at or close to full extension. Furthermore, it was found that the use of outer bandages having such limited extensibility aids in providing desirably low resting pressures and yet at the same time high walking pressures of the applied bandage system.

For further ease in application and avoidance of formation of wrinkling of the inner skin facing bandage during application of the bandage, it has been found beneficial to provide an inner bandage having a stretch capability of less than 75% (more particularly, less than 65%, and most particularly, less than 50%) in the longitudinal direction.

It also has been found particularly advantageous to configure and adapt the outer bandage and the inner bandage, such that in use, the inner and outer bandages remain adhered to one another under elastic extension, e.g., without the use of a fastening mechanism. In such embodiments, after application, the outer and inner bandages in principle act as a single bandaging entity—minimizing, if not eliminating, any potential of slippage and/or wrinkling between the two bandaging layers, and thus facilitating comfort for the patient as well as overall conformability of the complete, applied bandage system and uniformity of compressive pressure over extended periods of time.

In some embodiments, bandage systems of the present disclosure can provide effective and sustained therapeutic performance without application of any additional elongated bandages besides the inner and outer bandages described herein.

As mentioned above, in use of bandage systems described herein, the exposed face 14 of the foam layer 12 facing directly towards and coming into the contact with the skin of the patient demonstrates a particularly desirable and effective fastening onto the skin of the patient, which facilitates the minimization of tendencies of the bandage system towards slippage after application. To allow for desirable contouring of the foam layer to the particular limb of the patient and thus further enhanced, advantageous fastening of the foam onto the skin, the foam preferably has a thickness greater than 1.6 mm, more preferably greater than 2 mm. Within this range, a thickness of 10 mm or less is suitable; 8 mm or less being more suitable, 6 mm or less even more suitable, 5 mm or less yet even more suitable, 4 mm or less most suitable. To ensure such desirable fastening, the outer, exposed face 14 of the foam layer 12 is typically substantially free of materials, e.g., which could interfere with the foam-skin interface, being affixed to said face of the foam layer, such as fibers, nettings, and anti-adherent films. In some embodiments, the outer, exposed face 14 of the foam layer 12 (the face not affixed to substrate) does not comprise a self-adhering material. In other words, the outer, exposed face 14 of the foam layer 12 typically forms the innermost skin-facing surface of the inner bandage, with the possible exception of any optional tab material (typically having a width of 10% or less in the longitudinal direction of the bandage) at one or both terminal transverse ends of the bandage.

Bandage systems described herein may optionally include a wound dressing or plaster for covering and thus protecting an open wound, such as an ulcer, under the applied bandage system. Such dressings or plasters are typically appropriately sized to offer protection for the wound and immediate-surrounding skin about the wound. Such wound dressings or plaster are typically non-elongated. The term “non-elongated dressing or plaster” as used herein is generally understood to mean that the dressing or plaster is not sufficiently elongated so as to be capable of being wound two turns about a limb of a patient. In some embodiments, a non-elongated dressing or plaster is sized such that it can only be wound at most one turn about a limb of a patient, and in some embodiments, it is sized such that it cannot be wound one turn about a limb of a patient.

Bandage systems of the present disclosure can be provided in the form of a kit-of-parts.

Bandage systems of the present disclosure can be particularly adapted for use in the treatment and/or management of edema and other venous and lymphatic disorders of a limb, more particularly, venous leg ulcers and lymph edema of a limb.

In methods of using compression bandage systems described herein, the inner bandage is applied, e.g., by spirally winding the bandage about a limb of a patient, with the foam layer facing the skin of the patient, and subsequently the outer bandage is applied, e.g., again by spirally winding the bandage, over the inner bandage. If desired or needed, prior to the application of the inner bandage, a wound dressing or plaster may be applied to a wound or wounds.

The particular, appropriate dimensions of the bandages depend in part on the particular limb being treated and/or the particular patient. For example, in human (adult) therapy for use with lower limbs, suitable dimensions for the bandages may be about 70 to about 130 mm wide and about 2 to about 4.5 m long, while for use with upper limbs a width of about 70 to about 130 mm is suitable with a corresponding shorter length than that use for lower limbs. For applications in veterinary medicine, depending on the particular animal patient, appropriate, suitable dimensions may be larger (e.g., for equine bandaging) or smaller (e.g., for canine bandaging).

Each bandage is desirably, sufficiently porous to allow for transmission of air and moisture vapor through the bandage (e.g., a water vapor transmission rate (WVTR) of at least 240 g/m²/24 h, more suitably of at least 400 g/m²/24 h, e.g., as determined by ASTM E398-03 at 37.8° C. and 100% relative humidity in the wet chamber and 37.8° C. and 10% relative humidity in the dry chamber). In addition, each bandage, in particular the inner skin-facing bandage, may be sterilized, e.g., gamma sterilized.

Referring to FIG. 2, the outer, elongated, self-adhering elastic bandage 20 of compression bandage systems of the present disclosure can be adapted to provide a compressive force, more particularly a permanent compressive force, when extended.

In use, preferred outer bandages will provide a sub-bandage, resting compressive force of from about 1 to about 80 mm Hg (more suitably from about 20 to about 75 mmHg, most suitably from about 30 to about 70 mmHg) at a position 8 cm above the medial malleolus, when wrapped about a human adult leg with an ankle circumference of 22 cm.

As mentioned above, for ease in application and aiding in providing desirable low resting pressures and high walking pressures, it has been found particularly advantageous to provide outer elastic, compression bandages having a limited, relatively low extensibility in its longitudinal direction, in particular having a stretch capability in the longitudinal direction of not more than 75%, more preferably not more than 65%, most preferably not more than 55%, e.g., as determined in accordance with the Stretch Testing Procedure summarized below. Within this range, a minimal stretch capability of at least 20% in the longitudinal direction is desirable, at least 25% more desirable, and at least 30% most desirable. To ensure favorable conformability and retention of compressive recovery of the bandage through the time period the bandage is in place, the outer bandage desirably shows high elasticity in its longitudinal direction, in particular a recovery-of-stretch capability of at least 85%, more desirably at least 90%, most desirably at least 95%, in the longitudinal direction, e.g., as determined in accordance with the Stretch Testing Procedure summarized below.

Preferred outer bandages do not adhere to clothing, hair or skin.

Preferred outer bandages are self-adhering elastomeric bandages, more preferably self-adherent elastomeric bandages, which do not adhere to clothing, hair or skin.

Examples of suitable types of self-adherent elastomeric bandages as well as methods of making such bandages are disclosed in U.S. Pat. Nos. 3,575,782; 4,984,584; and US Application 2005/0025937A, which are incorporated herein by reference in their entirety. Other example of suitable types of self-adherent bandages include knitted and woven bandages commercially available under the trade designations ROSIDAL HAFT (Lohman & Rauscher GmbH & Co. KG, Neuwied Germany) and ACTICO (Activa Health Care, Burton-upon-Trent, UK).

As mentioned above, the outer bandage 20 can include a polymeric binder. Suitable polymeric binders providing cohesive properties may be either elastomeric or non-elastomeric polymeric binders, however, preferably the polymeric binder is an elastomeric polymeric binder due to generally favorable properties of such binders, such as long-term flexibility, extensibility and/or elasticity. Suitable elastomeric polymeric binders may comprise natural rubber latex, a synthetic latex, such as homopolymer and copolymer latexes of acrylics, butadienes, styrene/butadiene rubbers, chloroprenes, ethylenes (e.g., vinyl acetate/ethylene), isoprenes, nitriles and urethanes, or mixtures thereof. Examples of suitable polymeric elastomeric binders are disclosed, for example, in U.S. Pat. Nos. 3,575,782 and 4,984,585. Outer bandages may be desirably free of natural rubber latex.

For configurations including elastic yarns bound on a web or between webs, suitable webs include woven, knitted, warp-knit, or nonwoven fibrous webs, woven and nonwoven fibrous webs being more suitable, and nonwoven fibrous webs most suitable in terms of providing a favorably thin outer compression bandage, especially in its extended state. As mentioned above, preferably elastic yarns are partially extended (e.g., being maintained under partial tension) within the bandage. In order to provide preferred limited extensibility in the longitudinal direction as described above, during the manufacturing of such bandages (e.g., during binding of elastic yarns with polymeric binder between said webs or on a web) it is preferable to stretch the yarns to a length of at most 2.0, more preferably at most 1.75, even more preferable at most 1.5, most preferably about 1.5 times their fully relaxed length. The ratio of stretched length to relaxed length of yarn is referred to as draw ratio. Generally a draw ratio of at least 1.2 to 1 is desirable. Extent of compression provided is generally related to, inter alia, size of the elastic yarns and the number of yarns, whereby increased compression is typically a result of using greater number of larger elastic yarns in the bandage. Suitably, the number of elastic yarns per inch (epi) may range from about 8 to about 25 epi, while the elastic yarns may have a denier ranging from about 280 to about 1700. For use in bandage systems for treatment and/or management of venous leg ulceration, it has been found that the use of from about 10 to about 20 epi together with a elastic yarn denier of about 650 or less (more favorably about 620 or less, most favorably about 580 or less) in outer bandages is beneficial in providing desirable ease in handling of the outer bandage itself as well as desired therapeutic compressive force without observation of undesirable high resting pressures. Within the mentioned denier range, a suitable minimal denier for effective desired therapeutic compressive force may be at least about 350 denier (more favorably at least about 425 denier, and most favorably at least about 500 denier).

As mentioned above, for enhanced ease in application and avoidance of formation of wrinkling of the inner skin facing bandage during application of the bandage, it has been found preferably to provide an inner bandage having a stretch capability of less than 75% (more preferably less than 65%, most preferably less than 50%) in the longitudinal direction, e.g., as determined in accordance with the Stretch Testing Procedure summarized below. Within this range a minimal stretch capability of at least 15% in the longitudinal direction is desirable, at least 20% more desirable, and at least 25% most desirable. To ensure favorable conformability, the inner bandage desirably shows a recovery-of-stretch capability of at least 80%, more desirably at least 85%, most desirably at least 90%, in the longitudinal direction, e.g., as determined in accordance with the Stretch Testing Procedure summarized below.

Also as mentioned above, it also has been found particularly advantageous to configure and adapt the outer bandage and the inner bandage (in particular at least the outer face of the inner bandage (e.g., the face of the inner bandage facing away from the skin and towards the outer bandage in use)), such that in use the inner and outer bandages remain adhered to one another under elastic extension, e.g., without the use of a fastening mechanism. Such configurations may include an inner bandage, in particular its outer face, comprising the same self-adherent material as the outer bandage or another appropriate self-adherent material, such that in use the inner and outer bandages remained adhered to one another under elastic extension, e.g., without the use a fastening mechanism.

Desirably, the outer face of the inner bandage comprises a self-adhering material, more desirably, a self-adhering elastomeric material. The outer face of the inner bandage may be provided with such a self-adherent material, for example by providing (e.g., affixing) an elongated layer or a web including such material onto the second face 18 of the elastic substrate, i.e. the face of the elastic substrate opposite of the face (i.e. the first face) to which the foam layer is coupled. However, in consideration of providing favorably thin inner bandages and thus wearing comfort for the patient, the second face 18 of the elastic substrate 16 can form the outer face of the inner bandage as shown in FIG. 1. Accordingly, preferred embodiments of the inner bandage comprise an elastic substrate, in particular an elastic substrate in which at least its second face 18, comprises a self-adhering material, more preferably a self-adhering elastomeric material. In some embodiments in which at least the second face 18 of the elastic substrate is self-adherent, the outer bandage 20 may be eliminated, and the bandage system or compression dressing can include only the inner bandage 10, comprising an antimicrobial foam of the present disclosure.

The elastic substrate may favorably be made of a material (more favorably a self-adhering material, more favorably a self-adhering elastomeric material), which is capable of exerting a compressive force (in particular a permanent compressive force) when extended. In such preferred embodiments, although the elastic substrate may suitably be made of the same material as the outer bandage, it has been found more suitable to provide a related compression material that it provides a lesser amount of compression (than the outer bandage) when extended.

Elastic substrates may suitably comprise a woven, knitted or a nonwoven web comprising generally a plurality of generally longitudinally extending elastic yarns in the woven, knitted or nonwoven structure, said web being coated or impregnated with a polymer binder. More suitably elastic substrates of the inner bandage may comprise a plurality of generally longitudinally extending, partially extended or non-extended elastic yarns bound with a polymeric binder between two webs or bound with a polymeric binder on a web. In some embodiments, the polymeric binder is cohesive, so that elastic substrate is self-adherent, but will not adhere so clothing, hair or skin. Accordingly, at least the second face and more suitably both faces of the elastic substrate comprise polymeric binder (e.g., where the polymeric binder extends throughout the thickness of the web). Suitable polymeric binders include those described above in connection with outer bandages. Accordingly suitable polymeric binders providing cohesive properties may be either elastomeric or non-elastomeric polymeric binders. Preferably the polymeric binder is an elastomeric polymeric binder. Suitable elastomeric polymeric binders may comprise natural rubber latex, a synthetic latex, such as homopolymer and copolymer latexes of acrylics, butadienes, styrene/butadiene rubbers, chloroprenes, ethylenes (e.g., vinyl acetate/ethylene), isoprenes, nitriles and urethanes, or mixtures thereof. Again examples of suitable polymeric elastomeric binders are disclosed for example in U.S. Pat. Nos. 3,575,782 and 4,984,585. Inner bandages may be desirably free of natural rubber latex.

In embodiments of the inner bandage, in particular the elastic substrate thereof, including any type of self-adhering material (as described above), it is preferred that the respective self-adhering material does not adhere to clothing, hair or skin.

For configurations including elastic yarns bound on a web or between webs, suitable webs include woven, knitted, warp-knit, or nonwoven fibrous webs, woven and nonwoven fibrous webs being more suitable, and nonwoven fibrous webs most suitable in terms of providing a favorably thin elastic substrate, especially in its extended state. Partially extended yarns are preferred. During the manufacturing of such elastic substrates (e.g., during binding of elastic yarns with polymeric binder between said webs or on a web) it is preferable to stretch the yarns to a length of 5 times or less (more favorably 3.5 or less) times their fully relaxed length. Generally a draw ratio of at least 1.2 to 1 is desirable. Favorably the epi is less than 15, more favorably 12 or less, most favorably 10 epi or less. Within this range, an epi of 4 or more is suitable, 5 or more is more suitable, 6 or more is most suitable. Desirably elastic yarns have a denier less than 550, more desirably 450 or less, most desirably about 350 or less. Within this range, a denier of 100 or more is suitable, 150 or more is more suitable, and 200 or more is most suitable.

As shown in FIG. 1, the foam layer 12 is affixed to the first face of the elastic substrate. A variety of means are suitable for affixing the foam layer 12 onto the elastic substrate 16 such as stitching, needle tacking, ultrasonic welding or bonding, e.g., mechanical, thermal, and chemical bonding as well as combinations thereof. Suitable means of chemical bonding include using an adhesive, for example in the form of a continuous or discontinuous layer (e.g., a pattern-coated adhesive layer). Suitable adhesives for use can be any of those useful for wound dressings, such as those disclosed in WO 99/27975; WO 99/28539; U.S. Re. 24,906; U.S. Pat. No. 5,849,325; and U.S. Pat. No. 4,871,812; which are incorporated herein by reference in their entirety. Another suitable means of bonding includes providing the first face of the elastic substrate with a polymeric binder, in particular, an elastomeric polymer binder, having cohesive properties (as described above) and affixing the foam to the first face of the elastic substrate by applying the foam under pressure onto the substrate (e.g., passing the elongated foam and substrate through two driven rollers at a pressure around 0.3 M Pa), wherein a chemical and/or mechanical bond is provided between the foam and substrate. Alternatively the foam layer 12 may be affixed to the first face of the elastic substrate 16 by forming the foam directly onto the elastic substrate 16. To ensure a relatively smooth, generally non-wrinkled and/or non-puckered foam layer, preferably the foam layer is affixed to the elastic substrate, while the substrate is in a generally non-extended (e.g., 10% or less of the substrate total extensibility) state or a completely relaxed state.

Generally, the foam layer 12 is suitably affixed to the elastic substrate 16 beginning substantially at one transverse end of the substrate and extending 67% or more (more desirably 80% or more, more desirably 90% or more, even more desirably 95%) across the length of substrate towards the second transverse end. The portion near the second transverse end of the elastic substrate may be not covered by the foam layer, for example, to provide a tab of elastic substrate alone at the very end of the bandage to allow one or two wraps of elastic substrate onto itself. However in preferred embodiments, as shown in FIG. 1, the foam layer 12 is essentially coextensive or coextensive with the elastic substrate 16 face in the longitudinal direction. It can be preferable that the foam layer is essentially coextensive or coextensive in the longitudinal direction, because during bandaging, it is desired for therapeutic reasons and/or patient comfort, to have the person applying the bandage to simply cut off any excess bandage in length, and it has been observed that if the bandage includes a tab at the end, very often the applier, feeling obliged to make use of the tab, will not cut off any excess length.

Also generally the foam layer 12 is suitably affixed to the elastic substrate 16 beginning substantially at one longitudinal edge of the substrate and extending 33% or more across the width of the substrate towards the second longitudinal end. The particular amount of extension of the foam layer 12 across the width of the elastic substrate 16 (transverse extension) depends in part on how the inner bandage is applied. For example applications using a spiral winding of the inner bandage about a limb using standard 67% or 50% overlaps, a 33% and 50% transverse extension, respectively, may be suitable. Here, for example, as the bandage is spirally wound about the limb, the exposed face 14 of foam layer 12 comes into contact with the skin and the portion of the first inner face (along the length) of elastic substrate, which is not covered by the foam layer, comes into contact with the outer face of the inner bandage (from the previous turn). To further enhance ease in application and more importantly to facilitate uniformity of compressive force upon application of the bandage-system and the maintenance of a uniform compressive force over time, after application, it has been found advantageous to apply the inner bandage with an overlap of less than 50% (in particular with an overlap of 33% or less, more particular 20% or less, even more particular 10% or less, most particularly 5% or less). Accordingly the transverse extension of the foam layer is advantageously 50% or more (in particular 67% or more, more particular 80% or more, even more particular 90% or more, yet even more particular 95% or more). In preferred embodiments as shown in FIG. 1, the foam layer 12 is essentially coextensive or coextensive with the first face of the elastic substrate in the transverse direction.

Suitable foams may be either hydrophilic or hydrophobic, more suitably they may be hydrophobic and treated to render them more hydrophilic, e.g., with surfactants such as nonionic surfactants, such as oxypropylene-oxyethylene block copolymers.

As mentioned above, in use of bandage systems of the present disclosure, the exposed face 14 of the foam layer 12 facing directly towards and coming into the contact with the skin of the patient demonstrates a particularly desirable and effective fastening onto the skin of the patient, which facilitates the minimization of tendencies of the bandage system towards slippage after application. To allow for desirable contouring of the foam layer to the particular limb of the patient and thus further enhanced, advantageous fastening of the foam onto the skin, the foam preferably has a thickness greater than 1.6 mm, more preferably, greater than 2 mm Within this range, a thickness of 10 mm or less is suitable; 8 mm or less being more suitable, 6 mm or less even more suitable, 5 mm or less yet even more suitable, 4 mm or less most suitable. To ensure such desirable fastening, the outer, exposed face 14 of the foam layer 12 is typically substantially free of materials, e.g., which could interfere with the foam-skin interface, being affixed to said face of the foam layer, such as fibers, nettings, and anti-adherent films. In other words, the outer, exposed face 14 of the foam layer 12 typically forms the innermost skin-facing surface of the inner bandage, with the possible exception of any optional tab material (typically having a width of 10% or less in the longitudinal direction of the bandage) at one or both terminal transverse ends of the bandage.

In some embodiments, methods of using compression dressings or bandage systems of the present disclosure, after any optional application of a non-elongated wound dressing or plaster to cover any open wound or wounds and the immediate skin area surrounding such wound(s), can include applying the inner bandage 10 with foam layer 12 facing towards and contacting the skin, typically in a spiral technique as described above with an appropriate overlap. In some embodiments, the inner bandage 10 can be applied using a minimal amount of tension, or no tension. If necessary or desired, the inner bandage 10 can be temporarily fixed, e.g., at the end of the last wrap, using a piece of adhesive tape or another type of suitable fastener. Alternatively, but less preferable, a tab of adherent (preferably self-adherent) material may be added on the inner skin-facing face and terminal end of the inner bandage 10 in order to provide a suitable, integral fastening means for temporarily fastening the end of the last wrap of the inner bandage 10. Subsequently, the outer bandage 20 can be applied, again typically suitably in a spiral technique with an appropriate overlap (suitably a standard 50% overlap). The outer bandage 20 can applied under tension, preferably near or at full extension. For patients who cannot tolerate the desired therapeutic compressive force due to pain or over-sensitivity, it may be necessary or desirable to apply the outer bandage 20 with a lower degree of extension. In applications over joint areas, e.g., over an ankle, a figure-of-eight configuration may be used in combination with a spiral technique to ensure complete coverage. Once in place, the outer bandage 20 can hold the bandage system in place for extended periods of time to provide a therapeutic effect.

The following embodiments are intended to be illustrative of the present disclosure and not limiting.

### EMBODIMENTS

1. An antimicrobial foam comprising:
a polymeric foam, the foam having a wet-out time of at least 250 sec; and
a coating comprising an antimicrobial material comprising a quaternary ammonium compound, the antimicrobial material covalently bound to the foam;
wherein the antimicrobial foam comprises at least 0.25 parts by weight of quarternary ammonium compound per 100 parts by dry weight of the foam.

2. The antimicrobial foam of embodiment 1, wherein the antimicrobial foam comprises at least 0.5 parts by weight of quarternary ammonium compound per 100 parts by dry weight of the foam.

3. The antimicrobial foam of embodiment 1 or 2, wherein the antimicrobial foam comprises at least 1 part by weight of quarternary ammonium compound per 100 parts by dry weight of the foam.

4. The antimicrobial foam of any of embodiments 1-3, wherein the quaternary ammonium compound is polymeric.

5. The antimicrobial foam of any of embodiments 1-4, wherein the quaternary ammonium compound includes a silane-based quaternary ammonium compound.

6. The antimicrobial foam of embodiment 5, wherein the silane-based quaternary ammonium compound is covalently bonded to the foam via a silyl ether bond.

7. The antimicrobial foam of embodiment 5 or 6, wherein the silane-based quaternary ammonium compound covalently bound to the foam is derived from a reaction between a silane-based quaternary ammonium precursor and at least one of a hydroxyl group, an isocyanate group, and an amine group of the foam.

8. The antimicrobial foam of any of embodiments 5-7, wherein the silane-based quaternary ammonium compound is derived from a quaternary ammonium precursor having the formula:

(R¹O)₃SiR²N⁺R³R⁴(CH₂)ₙCH₃X⁻
wherein:
R¹ is selected from H, CH₃, or C₂H₅,
R² has the formula CₘH₂ₘ₊₁, where m is an integer ranging from 1 to 4,
R₃ and R₄ could be H, CH₃, C₂H₅,
n is an integer ranging from 2 to 22, and
X is selected from Cl, Br, BF₄, N(SO₂CF₃)₂, O₃SCF₃, and O₃SC₄F₉.

9. The antimicrobial foam of any of embodiments 5-8, wherein the silane-based quaternary ammonium compound is derived from 3-trimethoxysilylpropyldimethyloctadecyl ammonium chloride.

10. The antimicrobial foam of any of embodiments 1-7, wherein the quaternary ammonium compound includes a polyacrylate quaternary ammonium polymer.

11. The antimicrobial foam of embodiment 10, wherein the polyacrylate quaternary ammonium polymer is derived from a quaternary amine-functionalized ethylenically unsaturated monomer.

12. The antimicrobial foam of embodiment 10 or 11, wherein the polyacrylate quaternary ammonium polymer is derived from copolymerization of:
(i) a monomer that contains a quaternary ammonium compound, and
(ii) at least one monomer that contains a pendant group capable of reacting with the foam to form a covalent bond.

13. The antimicrobial foam of embodiment 12, wherein the quaternary ammonium compound is in a pendant group.

14. The antimicrobial foam of any of embodiments 10-13, wherein the polyacrylate quaternary ammonium polymer is derived from monomers, and wherein at least some of the monomers have the formula:

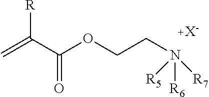

wherein:
R is selected from H and CH₃;
R⁵ and R⁶ are each selected from CH₃ and C₂H₅;
R⁷ is selected from CH₃, C₄H₉, C₆H₁₃, C₁₀H₂₁, C₁₂H₂₅, C₁₆H₃₃, C₁₈H₃₇, C₂₀H₄₁, an and C₂₂H₄₅;
X is selected from Cl, Br, BF₄, N(SO₂CF₃)₂, O₃SCF₃, and O₃SC₄F₉.

15. The antimicrobial foam of any of embodiments 10-14, wherein the polyacrylate quaternary ammonium polymer is derived from the polyacrylate quaternary ammonium compound having the formula:

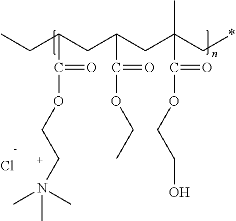

16. The antimicrobial foam of embodiment 15, wherein the polyacrylate quaternary ammonium compound includes a pendant —OH group, and wherein the polyacrylate quaternary ammonium compound is covalently bonded to the foam via a covalent bond formed between the pendant OH group and at least one of a hydroxyl group, an isocyanate group, and an amine group of the foam.

17. The antimicrobial foam of any of embodiments 10-16, wherein the polyacrylate quaternary ammonium polymer is derived from the polyacrylate quaternary ammonium compound having the formula:

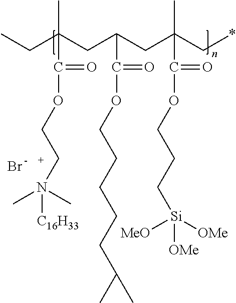

18. The antimicrobial foam of embodiment 17, wherein the polyacrylate quaternary ammonium compound includes a pendant —OR group, wherein the polyacrylate quaternary ammonium compound is covalently bonded to the foam via a covalent bond formed between the pendant —OR group and at least one of a hydroxyl group, an isocyanate group, and an amine group of the foam, and wherein R is a hydrogen, a methyl group, or an ethyl group.

19. The antimicrobial foam of any of embodiments 1-18, wherein the antimicrobial material is covalently bound to the foam via at least one of an ether bond, a silyl ether bond, an ester bond, a silyl ester bond, a urethane bond, a silyl urethane bond, an amide bond, and a silyl amide bond.

20. A method of making an antimicrobial foam, the method comprising:
providing a polymeric foam, the foam having a wet-out time of at least 250 sec;
providing an antimicrobial material comprising a quaternary ammonium precursor;
combining the foam and the quaternary ammonium precursor to form a combination; and
heating the combination to a temperature of at least 40° C. to form an antimicrobial foam comprising a quaternary ammonium compound covalently bound to the foam.

21. The method of embodiment 20, wherein heating initiates a reaction of the antimicrobial material with at least one of a hydroxyl group, an isocyanate group, and an amine group of the foam.

22. The method of embodiment 20 or 21, wherein heating initiates a reaction of an —OR group of the antimicrobial material with at least one of a hydroxyl group, an isocyanate group, and an amine group of the foam, wherein R is selected from H, CH₃ and C₂H₅.

23. The method of any of embodiments 20-22, wherein the antimicrobial foam comprises at least 0.25 parts by weight of quarternary ammonium compound per 100 parts by dry weight of the foam.

24. The method of any of embodiments 20-23, wherein the antimicrobial foam comprises at least 0.5 parts by weight of quarternary ammonium compound per 100 parts by dry weight of the foam.

25. The method of any of embodiments 20-24, wherein the antimicrobial foam comprises at least 1 part by weight of quarternary ammonium compound per 100 parts by dry weight of the foam.

26. The method of any of embodiments 20-25, wherein combining the foam and the quaternary ammonium precursor includes at least one of dipping, spraying, curtain coating, brushing, swabbing, padding, and a combination thereof.

27. The method of any of embodiments 20-26, wherein combining the foam and the quaternary ammonium precursor to form a combination and heating the combination occur at least partially simultaneously.

28. The method of any of embodiments 20-27, wherein the quaternary ammonium precursor includes a silane-based quaternary ammonium precursor.

29. The method of embodiment 28, wherein the silane-based quaternary ammonium compound covalently bound to the foam is derived from a reaction between the silane-based quaternary ammonium precursor and at least one of a hydroxyl group, an isocyanate group, and an amine group of the foam.

30. The method of embodiment 28 or 29, wherein the silane-based quaternary ammonium precursor has the formula:

(R¹O)₃SiR²N⁺R³R⁴(CH₂)ₙCH₃X⁻
wherein:
R¹ is selected from H, CH₃, or C₂H₅,
R² has the formula CₘH₂ₘ₊₁, where m is an integer ranging from 1 to 4,
R₃ and R₄ could be H, CH₃, C₂H₅,
n is an integer ranging from 2 to 22, and
X is selected from Cl, Br, BF₄, N(SO₂CF₃)₂, O₃SCF₃, and O₃SC₄F₉.

31. The method of any of embodiments 28-30, wherein the silane-based quaternary ammonium precursor is 3-trimethoxysilylpropyldimethyloctadecyl ammonium chloride.

32. The method of any of embodiments 20-29, wherein the quaternary ammonium precursor includes a polyacrylate quaternary ammonium compound, and wherein the polyacrylate quaternary ammonium compound comprises a polyacrylate quaternary ammonium monomer, oligomer or polymer.

33. The method of embodiment 32, wherein the polyacrylate quaternary ammonium compound includes or is derived from a quaternary amine-functionalized ethylenically unsaturated monomer.

34. The method of embodiment 32 or 33, wherein the polyacrylate quaternary ammonium compound includes or is derived from monomers, and wherein at least some of the monomers have the formula:

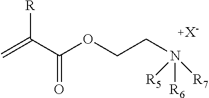

wherein:
R is selected from H and CH₃;
R⁵ and R⁶ are each selected from CH₃ and C₂H₅;
R⁷ is selected from CH₃, C₄H₉, C₆H₁₃, C₁₀H₂₁, C₁₂H₂₅, C₁₆H₃₃, C₁₈H₃₇, C₂₀H₄₁, and C₂₂H₄₅; and
X is selected from Cl, Br, BF₄, N(SO₂CF₃)₂, O₃SCF₃, and O₃SC₄F₉.

35. The method of any of embodiments 32-34, wherein the quaternary ammonium precursor includes a polyacrylate quaternary ammonium compound having the formula:

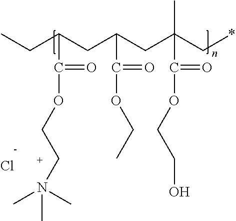

36. The method of embodiment 35, wherein the polyacrylate quaternary ammonium polymer includes a pendant —OH group, and wherein heating the combination initiates a reaction between the pendant —OH group of the polymer and at least one of a hydroxyl group, an isocyanate group, and an amine group of the foam.

37. The method of any of embodiments 32-36, wherein the quaternary ammonium precursor includes a polyacrylate quaternary ammonium compound having the formula:

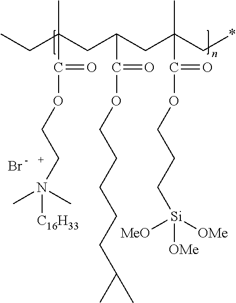

38. The method of embodiment 37, wherein the polyacrylate quaternary ammonium polymer includes a pendant methoxy group, and wherein heating the combination initiates a reaction between the pendant methoxy group of the polymer and at least one of a hydroxyl group, an isocyanate group, and an amine group of the foam.

39. The method of any of embodiments 20-38, wherein the quaternary ammonium compound is covalently bound to the foam via at least one of an ether bond, a silyl ether bond, an ester bond, a silyl ester bond, a urethane bond, a silyl urethane bond, an amide bond, and a silyl amide bond.

40. The antimicrobial foam of any of embodiments 1-19 or the method of any of embodiments 20-39, wherein the quaternary ammonium compound includes a silane-based quaternary ammonium compound, and wherein the silane-based quaternary ammonium compound is covalently bound to the foam.

41. The antimicrobial foam of any of embodiments 1-19 and 40 or the method of any of embodiments 20-40, wherein the quaternary ammonium compound includes a polyacrylate quaternary ammonium polymer, and wherein the polyacrylate quaternary ammonium polymer is covalently bound to the foam.

42. The antimicrobial foam of any of embodiments 1-19, 40 and 41 or the method of any of embodiments 20-41, wherein the quaternary ammonium compound is covalently bound to the foam via at least one of an ether bond, a silyl ether bond, an ester bond, a silyl ester bond, a urethane bond, a silyl urethane bond, an amide bond, and a silyl amide bond.

43. An antimicrobial foam comprising:
a polymeric foam; and
an antimicrobial material comprising a silane-based quaternary ammonium compound, wherein the silane-based quaternary ammonium compound is covalently bound to the foam;
wherein the antimicrobial foam comprises at least 1 part by weight of the silane-based quarternary ammonium compound per 100 parts by dry weight of the foam.

44. The antimicrobial foam of embodiment 43, wherein the silane-based quaternary ammonium compound covalently bound to the foam is derived from a reaction between at least one of a hydroxyl group, an isocyanate group, and an amine group of the foam and a silane-based quaternary ammonium precursor.

45. The antimicrobial foam of embodiment 43 or 44, wherein the silane-based quaternary ammonium compound is derived from a quaternary ammonium precursor having the formula:

(R¹O)₃SiR²N⁺R³R⁴(CH₂)ₙCH₃X⁻
wherein:
R¹ is selected from H, CH₃, or C₂H₅,
R² has the formula CₘH₂ₘ₊₁, where m is an integer ranging from 1 to 4,
R₃ and R₄ could be H, CH₃, C₂H₅,
n is an integer ranging from 2 to 22, and
X is selected from Cl, Br, BF₄, N(SO₂CF₃)₂, O₃SCF₃, and O₃SC₄F₉.

46. The antimicrobial foam of any of embodiments 43-45, wherein the silane-based quaternary ammonium compound is derived from 3-trimethoxysilylpropyldimethyloctadecyl ammonium chloride.

47. A method of making an antimicrobial foam, the method comprising:

providing a polymeric foam;

providing an antimicrobial material comprising a silane-based quaternary ammonium precursor;

combining the foam and the silane-based quaternary ammonium precursor to form a combination; and

heating the combination to a temperature of at least 40° C. to form an antimicrobial foam comprising a silane-based quaternary ammonium compound covalently bound to the foam.

48. The method of embodiment 47, wherein heating initiates a reaction of the silane-based quaternary ammonium precursor with at least one of a hydroxyl group, an isocyanate group, and an amine group of the foam.

49. The method of embodiment 47 or 48, wherein heating initiates a reaction of an —OR group of the silane-based quaternary ammonium precursor with at least one of a hydroxyl group, an isocyanate group, and an amine group of the foam, wherein R is selected from H, CH₃ and C₂H₅.

50. The method of any of embodiments 47-49, wherein the antimicrobial foam comprises at least 0.25 parts by weight of silane-based quaternary ammonium compound per 100 parts by dry weight of the foam.

51. The method of any of embodiments 47-50, wherein the antimicrobial foam comprises at least 0.5 parts by weight of silane-based quaternary ammonium compound per 100 parts by dry weight of the foam.

52. The method of any of embodiments 47-51, wherein the antimicrobial foam comprises at least 1 part by weight of silane-based quaternary ammonium compound per 100 parts by dry weight of the foam.

53. The method of any of embodiments 47-52, wherein combining the foam and the silane-based quaternary ammonium precursor includes at least one of dipping, spraying, curtain coating, brushing, swabbing, padding, and a combination thereof.

54. The method of any of embodiments 47-53, wherein combining the foam and the silane-based quaternary ammonium precursor to form a combination and heating the combination occur at least partially simultaneously.

55. The method of any of embodiments 47-54, wherein the silane-based quaternary ammonium precursor has the formula:

(R¹O)₃SiR²N⁺R³R⁴(CH₂)ₙCH₃X⁻
wherein:
R¹ is selected from H, CH₃, or C₂H₅,
R² has the formula CₘH₂ₘ₊₁, where m is an integer ranging from 1 to 4,
R₃ and R₄ could be H, CH₃, C₂H₅,
n is an integer ranging from 2 to 22, and
X is selected from Cl, Br, BF₄, N(SO₂CF₃)₂, O₃SCF₃, and O₃SC₄F₉.

56. The method of any of embodiments 47-55, wherein the silane-based quaternary ammonium precursor is 3-trimethoxysilylpropyldimethyloctadecyl ammonium chloride.

57. The antimicrobial foam of any of embodiments 43-46 or the method of any of embodiments 47-56, wherein the antimicrobial foam has a wet-out time of at least 250 sec.

58. The antimicrobial foam of any of embodiments 43-46 and 57 or the method of any of embodiments 47-57, wherein the silane-based quaternary ammonium compound is covalently bound to the foam via a silyl ether bond.

59. An antimicrobial foam comprising:
a polymeric foam; and
an antimicrobial material comprising a polyacrylate quaternary ammonium polymer, wherein the polyacrylate quaternary ammonium polymer is covalently bound to the foam;
wherein the antimicrobial foam comprises at least 1 part by weight of the quarternary ammonium compound per 100 parts by dry weight of the foam.

60. The antimicrobial foam of embodiment 59, wherein the polyacrylate quaternary ammonium polymer is derived from a quaternary amine-functionalized ethylenically unsaturated monomer.

61. The antimicrobial foam of embodiment 59 or 60, wherein the polyacrylate quaternary ammonium polymer is derived from monomers, and wherein at least some of the monomers have the formula:

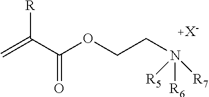

wherein:
R is selected from H and CH₃;
R⁵ and R⁶ are each selected from CH₃ and C₂H₅;
R⁷ is selected from CH₃, C₄H₉, C₆H₁₃, C₁₀H₂₁, C₁₂H₂₅, C₁₆H₃₃, C₁₈H₃₇, C₂₀H₄₁, and C₂₂H₄₅; and
X is selected from Cl, Br, BF₄, N(SO₂CF₃)₂, O₃SCF₃, and O₃SC₄F₉.

62. The antimicrobial foam of any of embodiments 59-61, wherein the polyacrylate quaternary ammonium polymer is derived from a polyacrylate quaternary ammonium compound having the formula:

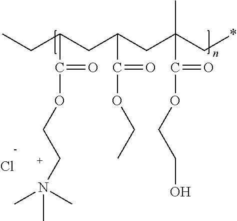

63. The antimicrobial foam of embodiment 62, wherein the polyacrylate quaternary ammonium compound includes a pendant —OH group, and wherein the polyacrylate quaternary ammonium polymer is covalently bonded to the foam via a covalent bond formed between the pendant —OH group of the polymer and at least one of a hydroxyl group, an isocyanate group, and an amine group of the foam.

64. The antimicrobial foam of any of embodiments 59-63, wherein the polyacrylate quaternary ammonium polymer is derived from a polyacrylate quaternary ammonium compound having at least one of a pendant hydroxyl group, a pendant alkoxyl group, a pendant carboxyl group, a pendant isocyanate group, and a pendant amine group.

65. The antimicrobial foam of any of embodiments 59-64, wherein the polyacrylate quaternary ammonium polymer is derived from a polyacrylate quaternary ammonium compound having the formula:

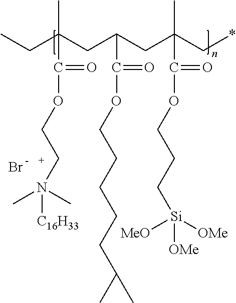

66. The antimicrobial foam of embodiment 65, wherein the polyacrylate quaternary ammonium compound includes a pendant —OR group, wherein the polyacrylate quaternary ammonium compound is covalently bonded to the foam via a covalent bond formed between the pendant —OR group and at least one of a hydroxyl group, an isocyanate group, and an amine group of the foam, and wherein R is a hydrogen, a methyl group or an ethyl group.

67. A method of making an antimicrobial foam, the method comprising:
providing a polymeric foam;
providing an antimicrobial material comprising a polyacrylate quaternary ammonium compound, wherein the polyacrylate quaternary ammonium compound comprises a polyacrylate quaternary ammonium monomer, oligomer or polymer;
combining the foam and the polyacrylate quaternary ammonium compound to form a combination; and
heating the combination to a temperature of at least 40° C. to form an antimicrobial foam comprising a polyacrylate quaternary ammonium polymer covalently bound to the foam.

68. The method of embodiment 67, wherein the polyacrylate quaternary ammonium compound comprises a polyacrylate quaternary ammonium monomer or oligomer, and wherein heating initiates:
polymerization of acrylate groups of the polyacrylate quaternary ammonium compound to form the polyacrylate quaternary ammonium polymer, and
reaction of the polyacrylate quaternary ammonium compound or polymer with the foam.

69. The method of embodiment 67 or 68, wherein heating initiates a reaction of an —OR group of the polyacrylate quaternary ammonium compound with at least one of a hydroxyl group, an isocyanate group, and an amine group of the foam, wherein R is selected from H, CH₃ and C₂H₅.

70. The method of any of embodiments 67-69, wherein the antimicrobial foam comprises more than 0.25 parts by weight of polyacrylate quarternary ammonium polymer per 100 parts by dry weight of the foam.

71. The method of any of embodiments 67-70, wherein the antimicrobial foam comprises more than 0.5 parts by weight of polyacrylate quarternary ammonium polymer per 100 parts by dry weight of the foam.

72. The method of any of embodiments 67-71, wherein the antimicrobial foam comprises more than 1 part by weight of polyacrylate quarternary ammonium polymer per 100 parts by dry weight of the foam.

73. The method of any of embodiments 67-72, wherein combining the foam and the polyacrylate quaternary ammonium compound includes at least one of dipping, spraying, curtain coating, brushing, swabbing, padding, and a combination thereof.

74. The method of any of embodiments 67-73, wherein combining the foam and the polyacrylate quaternary ammonium compound to form a combination and heating the combination occur at least partially simultaneously.

75. The method of any of embodiments 67-74, wherein the polyacrylate quaternary ammonium compound includes or is derived from a quaternary amine-functionalized ethylenically unsaturated monomer.

76. The method of any of embodiments 67-75, wherein the polyacrylate quaternary ammonium compound includes or is derived from monomers having the formula:

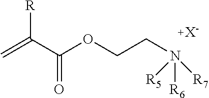

wherein:
R is selected from H and CH₃;
R⁵ and R⁶ are each selected from CH₃ and C₂H₅;
R⁷ is selected from CH₃, C₄H₉, C₆H₁₃, C₁₀H₂₁, C₁₂H₂₅, C₁₆H₃₃, C₁₈H₃₇, C₂₀H₄₁, and C₂₂H₄₅;
X is selected from Cl, Br, BF₄, N(SO₂CF₃)₂, O₃SCF₃, and O₃SC₄F₉.

77. The method of any of embodiments 67-76, wherein the polyacrylate quaternary ammonium compound includes a polyacrylate quaternary ammonium polymer having the formula:

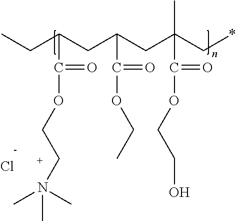

78. The method of embodiment 77, wherein the polyacrylate quaternary ammonium polymer includes a pendant —OH group, and wherein heating the combination initiates a reaction between the pendant —OH group of the polymer and at least one of a hydroxyl group, an isocyanate group, and an amine group of the foam.

79. The method of any of embodiments 67-78, wherein the polyacrylate quaternary ammonium compound includes a polyacrylate quaternary ammonium polymer having the formula:

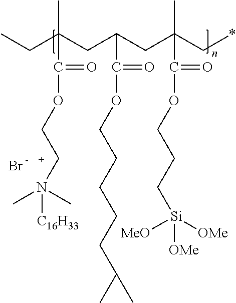

80. The method of embodiment 79, wherein the polyacrylate quaternary ammonium polymer includes a pendant —OR group, wherein heating the combination initiates a reaction between the pendant —OR group of the polyacrylate quaternary ammonium polymer and at least one of a hydroxyl group, an isocyanate group, and an amine group of the foam, and wherein R is a hydrogen, a methyl group, or an ethyl group.

81. The antimicrobial foam of any of embodiments 59-66 or the method of any of embodiments 67-80, wherein the antimicrobial foam has a wet-out time of at least 250 sec.

82. The antimicrobial foam of any of embodiments 59-66 and 81 or the method of any of embodiments 67-81, wherein the polyacrylate quaternary ammonium polymer is covalently bound to the foam via at least one of an ether bond, a silyl ether bond, an ester bond, a silyl ester bond, a urethane bond, a silyl urethane bond, an amide bond, and a silyl amide bond.

83. The antimicrobial foam of any of embodiments 59-66, 81 and 82 or the method of any of embodiments 67-82, wherein the polyacrylate quaternary ammonium polymer is covalently bound to the foam via at least one of an ether bond, a silyl ether bond, an ester bond, a silyl ester bond, a urethane bond, a silyl urethane bond, an amide bond, and a silyl amide bond.

84. The antimicrobial foam of any of embodiments 1-19, 40-46, 57-66 and 81-83 or the method of any of embodiments 20-42, 47-58 and 67-83, wherein the foam includes at least one of a polyurethane foam, a polyester foam and a polyether foam.

85. The antimicrobial foam of any of embodiments 1-19, 40-46, 57-66 and 81-84 or the method of any of embodiments 20-42, 47-58 and 67-84, wherein the antimicrobial foam forms at least a portion of a compression dressing.

86. The antimicrobial foam of any of embodiments 1-19, 40-46, 57-66, 81-85 or the method of any of embodiments 20-42, 47-58 and 67-85, wherein the antimicrobial foam forms at least a portion of a compression dressing, a wound dressing, and a combination thereof.

87. The antimicrobial foam of any of embodiments 1-19, 40-46, 57-66, 81-86 or the method of any of embodiments 20-42, 47-58 and 67-86, wherein the antimicrobial foam forms at least a portion of a comfort layer of a compression dressing.

88. The antimicrobial foam of any of embodiments 1-19, 40-46, 57-66, 81-87 or the method of any of embodiments 20-42, 47-58 and 67-87, wherein the antimicrobial material is non-leachable from the antimicrobial foam.

89. The antimicrobial foam of any of embodiments 1-19, 40-46, 57-66, 81-88 or the method of any of embodiments 20-42, 47-58 and 67-88, wherein the antimicrobial foam exhibits at least a 1 log reduction in either gram positive or gram negative bacteria, when tested pursuant to ASTM E2149-10.

90. A compression dressing comprising the antimicrobial foam of any of embodiments 1-19, 40-46, 57-66 and 81-89.

The following working examples are intended to be illustrative of the present disclosure and not limiting.

### EXAMPLES

### Materials

Materials used for the examples are shown in Table 1.

TABLE 1
Materials List

| Material | Description | Source |
|---|---|---|
| DMOAP | Dimethyloctadecyl[3- | Sigma Aldrich, St. Louis, MO |
| | (trimethoxysilyl)propyl]ammonium chloride, 42% | |
| | in methanol | |
| DMAEA-MCl | Dimethylaminoethyl acrylate methyl chloride | BASF located at Florham |
| | | Park, New Jersey |
| HEMA | Hydroxyethyl methacrylate | Cyro Industries, Parsippany, |
| | | NJ |
| Vazo-67 | 2,2-Azobis(2-methylbutyronitrile) | Dupont, Wilmington, DE |
| 1- | 1-Bromohexadecane | Chemtura Corporation, |
| Bromohexadecane | | Bay Minette, AL |
| DMAEMA | 2-(Dimethylamino)ethyl methacrylate, | CIBA, Marietta, GA |
| A-174 | Methacryloylpropyl trimethoxy silane | Sigma-Aldrich, St. Loius, MO |
| IOA | Iso-octyl acrylate | Sartomer USA, LLC; |
| | | Exton, PA |
| Quat-HEMA | p(DMAEA-MCl/EA/HEMA) (60/35/5) | 3M MRD |
| Quat-IOA | p(DMAEA-C₁₆Br/IOA/A-174) (50/40/10) | 3M MRD |
| Foam | Polyurethane foam layer from Coban ™ 2-Layer | 3M Company, St. Paul, MN |
| | Compression System | |
| P. aeruginosa | Pseudomonas aeruginosa, ATCC# 9027 | Microbiologics, Inc., St. |
| | | Cloud, MN |
| S. epidermidis | Staphylococcus epidermidis, ATCC# 14990 | Microbiologics, Inc., St. |
| | | Cloud, MN |
| BHI | Brain heart infusion broth | Becton, Dicksinson and |
| | | Company, Franklin Lakes, NJ |
| Sheep Blood | Defibrinated sheep blood | HemoStat Laboratories, |
| | | Dixon, CA |
| TSB | BBL ™ Tryptic soy broth | Becton-Dickenson, Sparks, |
| | | MD |
| Butterfield's buffer | Butterfield's buffer | 3M Company, St. Paul, MN |
| McFarland standard | Remel McFarland standard | Thermo Scientific, Lenexa, |
| | | KS |

### Test Methods

### Reduction of Microbial Growth

Reduction of microbial growth was determined based on ASTM E2149-10. Overnight cultures of bacteria were grown in TSB at 37° C. The bacteria were centrifuged and washed with Butterfield's buffer. The density was adjusted to ˜10⁸ in Butterfield's buffer using a 0.5 McFarland standard. A foam sample (approximately 1 gram) was placed into an Erlenmeyer flask follow by 50 mL of the bacteria suspension. The flask was placed in the incubator shaker for 1 hour at 250 RPM at 37° C. After 1 hour, the samples were serially diluted in phosphate buffer saline and plated on Petrifilm™ AC plates (3M Company, St. Paul, Minn.). The plates where incubated for 48 hours at 37° C. and the colonies counted. The growth reduction (Log CFU), as compared to an uncoated foam, was reported.

### Odor

Bacteria were grown overnight in TSB at 37° C. One microliter of this overnight culture was added to a 30 mL BHI/20 mL sheep blood mixture. Three paper towel disks were placed in the bottom of a 50 mL sterile tube. The paper towel disks were inoculated with 1 mL of the diluted bacteria solution. One foam sample was cut into a disk and placed on top of and in contact with the inoculated paper towel disks. The open tubes were placed in anaerobic chamber to remove oxygen and sealed. The tubes were then incubated in the anaerobic chamber at 37° C. for five days. The tubes were then removed, evaluated with a sniff test, and rated from 1 (minimal or no malodor) to 3 (significant malodor).

### Examples

### Synthesis of Quat-HEMA (Formula VII)

In a clean reaction bottle, 60 parts DMAEA-MC1 monomer, 35 parts ethyl acetate, and 5 parts HEMA monomer were combined with 0.5 parts Vazo-67 and 200 parts of isopropyl alcohol. The mixture was purged with dry nitrogen for 3 minutes. The reaction bottle was sealed, placed in a 65° C. water bath, and mixed for 17 hours. An additional 0.1 parts Vazo-67 was added to the mixture and the bottle purged and sealed. The bottle was placed in the 65° C. water bath and mixed for 8 hours. To this polymer solution was added 200 parts of DI water and the solution mixed for five minutes. The solution was heated to 70° C. under vacuum to remove solvent. When cooled, a 33% solids solution was obtained.

### Synthesis of DMAEMA-C₁₆Br Monomer

A clean reactor fitted with over head condenser, mechanical stirrer, and temperature probe was charged with 918 parts of acetone, 807 parts of 1-bromohexadecane, 415.5 parts of DMAEMA, 2.0 parts of BHT, and 2.0 parts of MEHQ. The batch was stirred at 150 rpm and 90/10 O₂/N₂ was purged through the solution. The mixture was heated to 74° C. for 18 hours followed by addition of 918 parts EtOAc with stirring at high speed. Heat was removed and the solution was allowed to cool to room temperature. The precipitated white solid was isolated by filtration and washed with 200 parts of cold EtOAc and dried in a vacuum oven at 40° C. for 8 hours. This was the DMAEMA-C₁₆Br monomer.

### Synthesis of Quat-IOA (Formula VIII)

In a clean reaction bottle, the monomers, 50 parts DMAEMA-C₁₆Br monomer, 10 parts A-174 monomer, and 40 parts IOA monomer were combined with 0.5 parts Vazo-67 and 300 parts isopropyl alcohol. The mixture was purged with dry nitrogen for 3 minutes. The reaction bottle was sealed, placed in a 65° C. water bath, and mixed for 17 hours. An additional 0.1 parts Vazo-67 was added to the mixture and the bottle purged and sealed. The bottle was placed in a 65° C. water bath and mixed for 8 hours.

### Example-1 (E-1)

DMOAP (Formula II) was diluted in water to 2%. A pre-weighed foam sample was placed in the diluted DMOAP solution for 1 to 5 minutes. The foam sample was then removed and the excess solution squeezed from the foam with a 2 kg roller. The foam was then dried at 70° C. and weighed to determine the antimicrobial uptake in grams/square meter.

### E-2 through E-13

E-2 through E-13 were prepared as described in E-1 as described in Table 2.

### E-14 and E-15

E-14 was prepared by preparing a 2% solids solution of quat-HEMA in water. A pre weighed foam sample was dipped into the solution for 2-5 minutes. The foam sample was then removed and the excess solution squeezed from the foam with a 2 kg roller. The foam was then dried in oven at 105° C. for 20-30 minutes. E-15 was prepared as described for E-14 using quat-IOA instead of quat-HEMA.

TABLE 2
Examples

| | Antimicrobial Solution | Antimicrobial Concentration |
|---|---|---|
| Sample | Concentration (%) | (parts/100 parts dry foam) |
| E-1 | 2.0 | 18.1 |
| E-2 | 2.0 | 11.6 |
| E-3 | 1.5 | 10.0 |
| E-4 | 1.5 | 7.3 |
| E-5 | 1.0 | 9.4 |
| E-6 | 1.0 | 0.2 |
| E-7 | 0.75 | 7.3 |
| E-8 | 0.75 | 2.9 |
| E-9 | 1.0 | 8.4 |
| E-10 | 0.75 | 4.8 |
| E-11 | 0.75 | 4.5 |
| E-12 | 0.50 | 9.4 |
| E-13 | 0.50 | 8.9 |
| E-14 | 2.0 | 4.9 |
| E-15 | 2.0 | 7.0 |

Example foam samples were tested for reduction of microbial growth and odor. Results are shown in Tables 3 through 5.

TABLE 3
Reduction of antimicrobial growth

| | S. epidermidis | P. aeruginosa |
|---|---|---|
| Sample | (Log CFU) | (Log CFU) |
| Control[a] | 8.5 | 8.5 |
| E-1 | 2 | 2 |
| E-2 | 2 | 2 |
| E-3 | 2 | 2 |
| E-4 | 2 | 2 |
| E-5 | 2 | 2 |
| E-6 | 2 | 2 |
| E-7 | 2 | 2 |
| E-8 | 2 | 2 |

[a]uncoated foam

TABLE 4
Reduction of antimicrobial growth

| | S. epidermidis | P. aeruginosa |
|---|---|---|
| Sample | (Log CFU) | (Log CFU) |
| Control[a] | 7.1 | 7.0 |
| E-9 | 0 | 0 |
| E-11 | 0 | 0 |
| E-13 | 0 | 0 |

[a]uncoated foam

TABLE 5
Odor

| Sample | S. epidermidis | P. aeruginosa |
|---|---|---|
| Control[a] | 3 | 3 |
| E-2 | 1 | 1 |
| E-10 | 1 | 1 |
| E-12 | 1 | 2 |
| E-14 | 1 | 1 |
| E-15 | 1 | 1 |

[a]uncoated foam

The embodiments described above and illustrated in the figures are presented by way of example only and are not intended as a limitation upon the concepts and principles of the present disclosure.

All references and publications cited herein are expressly incorporated herein by reference in their entirety into this disclosure.

Various features and aspects of the present disclosure are set forth in the following claims.

1. An antimicrobial foam comprising:
a polymeric foam, the foam having a wet-out time of at least 250 sec; and
a coating comprising an antimicrobial material comprising a quaternary ammonium compound, the antimicrobial material covalently bound to the foam;
wherein the antimicrobial foam comprises at least 0.25 parts by weight of quarternary ammonium compound per 100 parts by dry weight of the foam.

2. The antimicrobial foam of claim 1, wherein the antimicrobial foam comprises at least 0.5 parts by weight of quarternary ammonium compound per 100 parts by dry weight of the foam.

3. The antimicrobial foam of claim 1, wherein the antimicrobial foam comprises at least 1 part by weight of quarternary ammonium compound per 100 parts by dry weight of the foam.

4. The antimicrobial foam of claim 1, wherein the quaternary ammonium compound includes a silane-based quaternary ammonium compound.

5. The antimicrobial foam of claim 4, wherein the silane-based quaternary ammonium compound is covalently bonded to the foam via a silyl ether bond.

6. The antimicrobial foam of claim 4, wherein the silane-based quaternary ammonium compound covalently bound to the foam is derived from a reaction between a silane-based quaternary ammonium precursor and at least one of a hydroxyl group, an isocyanate group, and an amine group of the foam.

7. The antimicrobial foam of claim 4, wherein the silane-based quaternary ammonium compound is derived from a quaternary ammonium precursor having the formula:
(R¹O)₃SiR²N⁺R³R⁴(CH₂)ₙCH₃X⁻
wherein:
R¹ is selected from H, CH₃, or C₂H₅,
R² has the formula CₘH₂ₘ₊₁, where m is an integer ranging from 1 to 4,
R₃ and R₄ could be H, CH₃, C₂H₅,
n is an integer ranging from 2 to 22, and
X is selected from Cl, Br, BF₄, N(SO₂CF₃)₂, O₃SCF₃, and O₃SC₄F₉.

8. The antimicrobial foam of claim 4, wherein the silane-based quaternary ammonium compound is derived from 3-trimethoxysilylpropyldimethyloctadecyl ammonium chloride.

9. The antimicrobial foam of claim 1, wherein the quaternary ammonium compound includes a polyacrylate quaternary ammonium polymer.

10. The antimicrobial foam of claim 9, wherein the polyacrylate quaternary ammonium polymer is derived from a quaternary amine-functionalized ethylenically unsaturated monomer.

11. The antimicrobial foam of claim 9, wherein the polyacrylate quaternary ammonium polymer is derived from copolymerization of:
(i) a monomer that contains a quaternary ammonium compound, and
(ii) at least one monomer that contains a pendant group capable of reacting with the foam to form a covalent bond.

12. The antimicrobial foam of claim 9, wherein the polyacrylate quaternary ammonium polymer is derived from monomers, and wherein at least some of the monomers have the formula: 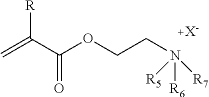
wherein:
R is selected from H and CH₃;
R⁵ and R⁶ are each selected from CH₃ and C₂H₅;
R⁷ is selected from CH₃, C₄H₉, C₆H₁₃, C₁₀H₂₁, Cl₂H₂₅, C₁₆H₃₃, C₁₈H₃₇, C₂₀H₄₁, and C₂₂H₄₅; and
X is selected from Cl, Br, BF₄, N(SO₂CF₃)₂, O₃SCF₃, and O₃SC₄F₉.

13. The antimicrobial foam of claim 9, wherein the polyacrylate quaternary ammonium polymer is derived from the polyacrylate quaternary ammonium compound having the formula:

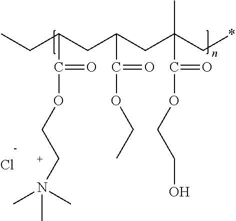

14. The antimicrobial foam of claim 13, wherein the polyacrylate quaternary ammonium compound includes a pendant —OH group, and wherein the polyacrylate quaternary ammonium compound is covalently bonded to the foam via a covalent bond formed between the pendant —OH group and at least one of a hydroxyl group, an isocyanate group, and an amine group of the foam.

15. The antimicrobial foam of claim 9, wherein the polyacrylate quaternary ammonium polymer is derived from the polyacrylate quaternary ammonium compound having the formula:

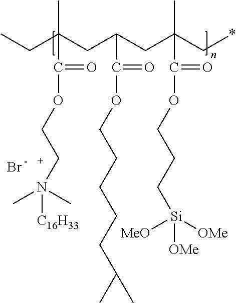

16. The antimicrobial foam of claim 15, wherein the polyacrylate quaternary ammonium compound includes a pendant —OR group, wherein the polyacrylate quaternary ammonium compound is covalently bonded to the foam via a covalent bond formed between the pendant —OR group and at least one of a hydroxyl group, an isocyanate group, and an amine group of the foam, and wherein R is a hydrogen, a methyl group, or an ethyl group.

17. The antimicrobial foam of claim 1, wherein the antimicrobial material is covalently bound to the foam via at least one of an ether bond, a silyl ether bond, an ester bond, a silyl ester bond, a urethane bond, a silyl urethane bond, an amide bond, and a silyl amide bond.

18. A compression dressing comprising the antimicrobial foam of claim 1.

19. A method of making an antimicrobial foam, the method comprising:
providing a polymeric foam, the foam having a wet-out time of at least 250 sec;
providing an antimicrobial material comprising a quaternary ammonium precursor;
combining the foam and the quaternary ammonium precursor to form a combination; and
heating the combination to a temperature of at least 40° C. to form an antimicrobial foam comprising a quaternary ammonium compound covalently bound to the foam.

20. The antimicrobial foam of claim 1, wherein the antimicrobial foam exhibits at least a 1 log reduction in either gram positive or gram negative bacteria, when tested pursuant to ASTM E2149-10.
